(19) United States
(12) Patent Application Publication
(10) Pub. No.: US 2021/0161365 A1
(43) Pub. Date: Jun. 3, 2021

(54) USER TERMINAL AND CONTROL METHOD OF THE SAME

(71) Applicant: SAMSUNG ELECTRONICS CO., LTD., Suwon-si (KR)

(72) Inventors: Do-young KIM, Hwaseong-si (KR); Hee-jin PARK, Seoul (KR); June-hyeon AHN, Suwon-si (KR); Sun-tae JUNG, Yongin-si (KR); Jae-geol CHO, Yongin-si (KR); Jin-sung KIM, Seoul (KR)

(73) Assignee: SAMSUNG ELECTRONICS CO., LTD., Suwon-si (KR)

(21) Appl. No.: 16/071,174

(22) PCT Filed: Dec. 29, 2016
(86) PCT No.: PCT/KR2016/015442

§ 371 Date: Jul. 19, 2018

(30) Foreign Application Priority Data
Jan. 19, 2016 (KR) 10-2016-0006672

(51) Int. Cl. A61B 1/00; A61B 1/24; A61B 1/045; A61B 1/04; A61B 1/06

(52) U.S. Cl. CPC A61B 1/00039; A61B 1/24; A61B 1/045; A61B 1/00006; A61B 1/00048; A61B 5/08; A61B 1/043; A61B 1/00032; A61B 1/00186; A61B 1/0661; A61B 1/00016; A61B 1/00009

(57) ABSTRACT

A user terminal of the present invention includes a display including a screen on which an image is displayable, and configured to output light having a specific wavelength from at least a partial region of the screen; an image capturer configured to capture an image of a user's body; and a controller configured to control the display to output first light of a first wavelength range from a first region on the screen, control the image capturer to capture an image, analyze the captured image, and control the display to display analysis information on a second region of the screen.

### TECHNICAL FIELD

The present invention relates to a user terminal and a control method of the same, in which light of a specific range is output to a body of a user and an image is captured based on reflected light, thereby providing analysis information about the user body.

### BACKGROUND ART

As a system related to the present invention, a personal terminal is used to capture a body part of a user, and a remote tooth diagnosis server is used to transmit the captured image to an expert. Then, the expert determines a conditions of teeth based on the received captured image and transmits medical information to a personal terminal.

FIG. 1 is a view of illustrating a remote tooth diagnosis system related to the present invention. A remote tooth diagnosis server 1 transmits a captured image of teeth (image A) received from a personal terminal 2 to a tooth expert terminal 3, and transmits diagnosis data from the tooth expert terminal 3 to the personal terminal 2. In FIG. 1, a user of the personal terminal 2 captures an image of teeth and transmits the captured image the remote tooth diagnosis server 1 so that the captured image can be delivered to the tooth expert terminal 3, and the remote tooth diagnosis server 1 transmits the captured image to the expert terminal 3. A dentist, who is not only a user of the expert terminal 3 but also an expert in tooth, reads the captured image with the naked eye and writes out teeth conditions of the user of the personal terminal 2 and diagnosis information, thereby transmitting the teeth conditions and the diagnosis information to the remote tooth diagnosis server 1 so as to be transmitted to the personal terminal 2. Thus, the user of the personal terminal 2 can receive the conditions of his/her teeth and the diagnosis information.

To receive a tooth diagnosis service provided as shown in FIG. 1, a user has to capture and transmit his/her teeth image for consulting the expert in tooth and cannot get information about his/her teeth until a predetermined period of time elapses. Further, it may be inconvenient for a user to determine next steps to be taken with a lack of received invention.

### DISCLOSURE

### Technical Problem

An object of the present invention is to provide a user terminal and a control method of the same, in which light of a specific range is output to a body of a user, an image is captured based on reflected light, and the capture image is analyzed to provide analysis information about the user body through a screen of the user terminal.

### Technical Solution

A user terminal conceived to solve the problems of the present invention includes: a display including a screen on which an image is displayable, and configured to output light having a specific wavelength from at least a partial region of the screen; an image capturer configured to capture an image of a user's body; and a controller configured to control the display to output first light of a first wavelength range from a first region on the screen, control the image capturer to capture an image, analyze the captured image, and control the display to display analysis information on a second region of the screen.

Here, the user terminal may further including a sensor configured to sense at least one of gas and temperature in the user's oral cavity, wherein the controller controls the display to output the first light based on a level of at least one of the gas and temperature sensed by the sensor

Further, the controller may analyze an image based on second light of a second wavelength range within the captured image.

Here, the controller may analyze at least one of color, distribution, illumination, fluorescence loss and area of the image based on the second light in accordance with the captured image.

Further, the controller may set a range of the at least one analyzed value for providing the analysis information.

Here, the controller may extract an image based on the second light from the captured image.

Further, the user terminal may further include: a power supply configured to supply power; and an optical filter configured to receive power from the power supply and operate to pass the second light but block light other than the second light of the reflected light caused by the first light.

Here, the controller may control the power supply so that the optical filter can start the operations when the first light is output.

Further, the controller may control at least one among a size and a position of the first region and brightness of the first light to be varied depending on at least one among a size, illumination and brightness of the image captured with the second light.

Here, the user terminal may further include: a storage configured to store an application program for determining a state of a user's oral cavity; and a user interface (UI) generator configured to generate a UI for the application program, wherein the controller executes the application program, and controls the UI generator to generate a UI selectable for measuring at least one among dental plaque, dental caries, bad breath and oral temperature of the user.

Further, the analysis information may include at least one among a user's oral state information, oral care information, education and coaching information for an oral cavity, goal setting challenge information, goal attainment guide information and guide information.

Here, the display may include an RGB filter for outputting the first light.

Further, the image based on the second light may have green and red fluorescent colors reflected and received after outputting the first light of blue color to secretions of germs in the user's oral cavity.

Meanwhile, a method of controlling a user terminal conceived to solve the problems of the present invention includes: controlling a first region on a screen of a display to output first light of a first wavelength range; capturing an image; analyzing the captured image; and displaying analysis information in a second region on the screen. Here, the controlling of the output of the first light may include controlling the first light to be output based on a level of at least one of gas and temperature sensed in a user's oral cavity.

Here, the controlling of the output of the first light may include controlling the first light to be output based on a level of at least one of gas and temperature sensed in a user's oral cavity.

Further, the analyzing of the captured image may include analyzing an image based on second light of a second wavelength range within the captured image.

Here, the displaying of the analysis information may include analyzing at least one of color, distribution, illumination, fluorescence loss and area of the image based on the second light in accordance with the captured image.

Here, the analyzing of the at least one may include setting a range of the at least one analyzed value for providing the analysis information.

Further, the analyzing of the captured image may include extracting an image based on the second light from the captured image.

Here, the method may further include supplying power to an optical filter to pass the second light but block light other than the second light of the reflected light.

Further, the supplying of the power may include controlling the optical filter to start the operations when the first light is output.

Here, the displaying of the analysis information may include controlling at least one among a size and a position of the first region and brightness of the first light to be varied depending on at least one among a size, illumination and brightness of the image captured with the second light.

Further, the method may further include executing the application program; and generating a UI selectable for measuring at least one among dental plaque, dental caries, bad breath and oral temperature of the user.

Here, the analysis information may include at least one among a user's oral state information, oral care information, education and coaching information for an oral cavity, goal setting challenge information, goal attainment guide information and guide information.

Further, the image based on the second light may have green and red fluorescent colors reflected and received after outputting the first light of blue color to secretions of germs in the user's oral cavity.

Meanwhile, an oral care apparatus conceived to solve the problems of the present invention includes: a communicator configured to communicate with a user terminal; a light emitter configured to output light having a specific wavelength to a user's oral cavity; an image capturer configured to capture an image of the user's oral cavity; and a controller configured to control the light emitter to output first light of a first wavelength range, control the image capturer to capture an image, and control the communicator to transmit the captured image to the user terminal.

Here, the oral care apparatus may further including a sensor configured to sense at least one of gas and temperature in the user's oral cavity, wherein the controller controls the display to output the first light based on a level of at least one of the gas and temperature sensed by the sensor

Further, the controller may control at least one between sensing at least one of gas and temperature in the user's oral cavity and outputting the first light, based on a control signal received from the user terminal.

Meanwhile, a method of controlling an oral care apparatus conceived to solve the problems of the present invention includes: outputting first light of a first wavelength range; capturing an image; and transmitting the captured image to a user terminal.

Here, the method may further include a level of at least one of gas and temperature sensed in the user's oral cavity to the user terminal.

Further, the outputting of the first light may include performing at least one between sensing at least one of gas and temperature in the user's oral cavity and outputting the first light, based on a control signal received from the user terminal.

### Advantageous Effects

According to the present invention, a user can easy get information about a state of teeth, diagnosis information, education information, coaching information, and goal guide information through his/her user terminal.

### DESCRIPTION OF DRAWINGS

FIG. 1 is a schematic view of illustrating a remote tooth diagnosis system related to the present invention,

FIG. 2 illustrates an example that a user terminal according to one embodiment of the present invention is used to capture teeth and analysis information about the teeth is displayed on a screen,

FIG. 3 is a block diagram of a user terminal according to the present invention,

FIG. 4 is a block diagram of a user terminal according to another embodiment of the present invention

FIG. 5 is a control flowchart of showing a control method of a user terminal according to the present invention,

FIG. 6 is a control flowchart of showing a control method of a user terminal according to one embodiment of the present invention,

FIG. 7 is a control flowchart of showing a control method of a user terminal according to another embodiment of the present invention,

FIG. 8 illustrates an example of showing a user interface (UI) for selecting a user's dental plaque and dental caries in a user terminal according to another embodiment of the present invention,

FIG. 9 is a graph of showing a screen spectrum of outputting blue light in a user terminal according to one embodiment of the present invention,

FIG. 10 illustrates an example of showing analysis information about a user body on a captured image in a user terminal according to one embodiment of the present invention,

FIGS. 11 and 12 illustrate examples of analyzing a color and an area through a grid region set on a captured image in a user terminal according to one embodiment of the present invention,

FIG. 13 illustrates an example of providing state information and diagnostic guide information on a captured image in a user terminal according to one embodiment of the present invention,

FIG. 14 is a flowchart of showing a control flow for measuring a fluorescence loss and providing analysis information about dental plaque in a user terminal according to one embodiment of the present invention,

FIG. 15 illustrates an example of the analysis information obtained from the control flow of FIG. 14,

FIG. 16 is a flowchart of showing a control flow for measuring an area of second light and providing analysis information about dental caries in a user terminal according to another embodiment of the present invention,

FIG. 17 illustrates an example of the analysis information obtained from the control flow of FIG. 16,

FIG. 18 illustrates an example of displaying guide information on a second region in a user terminal according to one embodiment of the present invention,

FIGS. 19 and 20 illustrate examples of showing a first region and a second region on a screen of a display (110) in a user terminal (10) according to still another embodiment of the present invention,

FIG. 21 is a block diagram of an oral care apparatus according to the present invention,

FIG. 22 is a block diagram of an oral care apparatus according to one embodiment of the present invention,

FIG. 23 is a flowchart of showing operations of an oral care apparatus according to the present invention,

FIG. 24 is a flowchart of showing operations of an oral care apparatus according to one embodiment of the present invention,

FIG. 25 is a flowchart of showing a control flow of providing analysis information based on measurement of a user's bad breath, oral temperature and light in an oral care apparatus and a user terminal according to one embodiment of the present invention,

FIG. 26 is a table of showing guide information provided to a user in a user terminal by measuring an oral temperature level of the user in an oral care apparatus according to one embodiment of the present invention,

FIG. 27 is a table of showing guide information provided to a user in a user terminal by measuring a bad breath level of the user in an oral care apparatus according to one embodiment of the present invention,

FIG. 28 illustrates an example that analysis information is provided by measuring a user's bad breath, oral temperature and light through a user terminal and an oral care apparatus according to one embodiment of the present invention,

FIGS. 29 to 31 illustrate examples of showing mounting positions and shapes of a light emitter, an image capturer and a sensor in an oral care apparatus according to one embodiment of the present invention,

FIG. 32 illustrates an example of showing a signal flow in measuring a user's bad breath, oral temperature and light through a user terminal and an oral care apparatus according to one embodiment of the present invention and providing analysis information,

FIGS. 33 and 34 illustrate examples of conducting a dental health education and coaching through an animation or a user set character for comparison between before and after brushing teeth with respect to measured results,

FIG. 35 illustrates an extended example that measured results for dental care are transmitted to a server capable of analyzing the measured results and services like those of FIGS. 33 and 34 are received through the server,

FIGS. 36 and 37 are flowcharts of showing a control method of a user terminal according to one embodiment of the present invention with regard to FIGS. 33 and 34, and

FIG. 38 illustrates an example of showing operations for providing analysis information about teeth through a user terminal and a server according to another embodiment of the present invention.

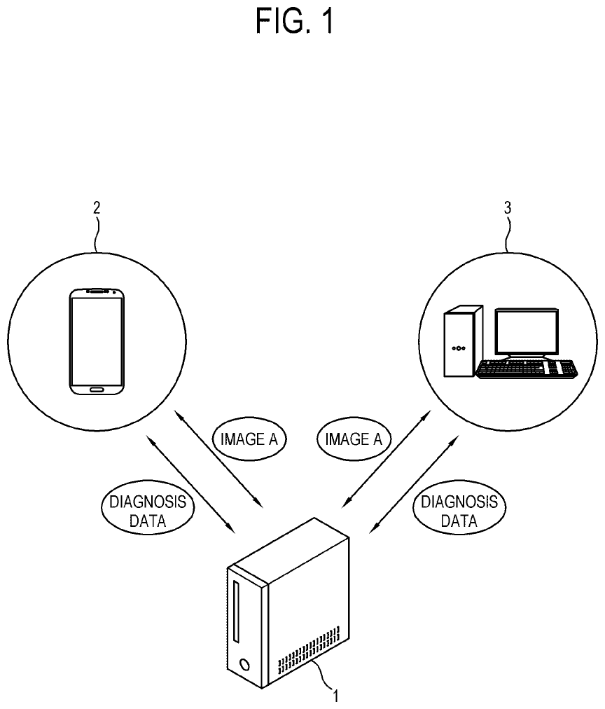

### BEST MODE

Below, the present invention will be described in detail with reference to accompanying drawings.

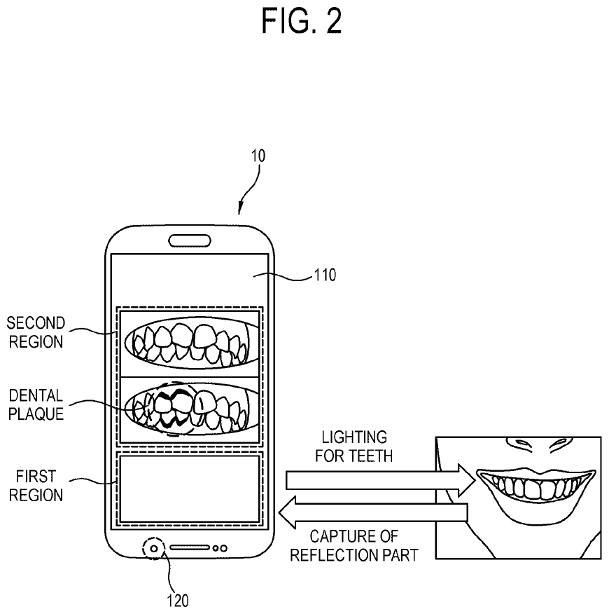

FIG. 2 illustrates an example that a user terminal 10 according to one embodiment of the present invention is used to capture teeth and analysis information about the teeth is displayed on a screen,

In the user terminal 10 shown in FIG. 2, a screen of a display 110 may be divided into a first region and a second region. The screen for the first region may be set in the display 110 so that first light, i.e. blue light of 400˜450 nm can be output to a user's oral cavity. Further, the screen of the second region may be set to display analysis information about a user's oral cavity, which is obtained by capturing an image through an image capturer 120 with second light reflected by projecting the first light output from the first region onto the user's oral cavity, and analyzing the captured image. The second region may display an image of oral cavity, i.e. show plaque building up on a user's teeth. When an image of a user's oral cavity is captured in such a state that a blue screen is displayed on the first region so that the blue light can be projected onto the user's oral cavity, the captured image includes an image based on the second light reflected by projecting the blue light onto the user's oral cavity, and it is thus possible to determine the state of the oral cavity by analyzing the image based on the second light in the captured image. When a user's oral cavity has a lot of tooth germs, there are many secretions from the tooth germs. Since light reflected by projecting the first light, i.e. the blue light onto the secretions of the tooth germs has a red fluorescent color, it is possible to determine how many tooth germs are there by analyzing the red fluorescent color from the image based on the second light in the captured image. In this example, the user terminal 10 captures a user's teeth, analyzes the teeth, and provides analysis information through the second region, but the present invention is not limited to this example. Alternatively, the user terminal 10 may capture an image based on the first light in the first region and transit data of the captured image to an external device, and the external device may analyze the data and transmit analysis information to the user terminal 10 so that the analysis information can be displayed in the second region of the user terminal 10.

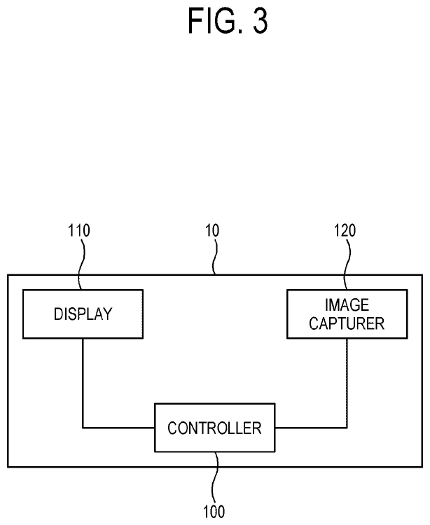

FIG. 3 is a block diagram of the user terminal 10 according to the present invention. The user terminal according to the present invention includes a display 110, an image capturer 120, and a controller 100.

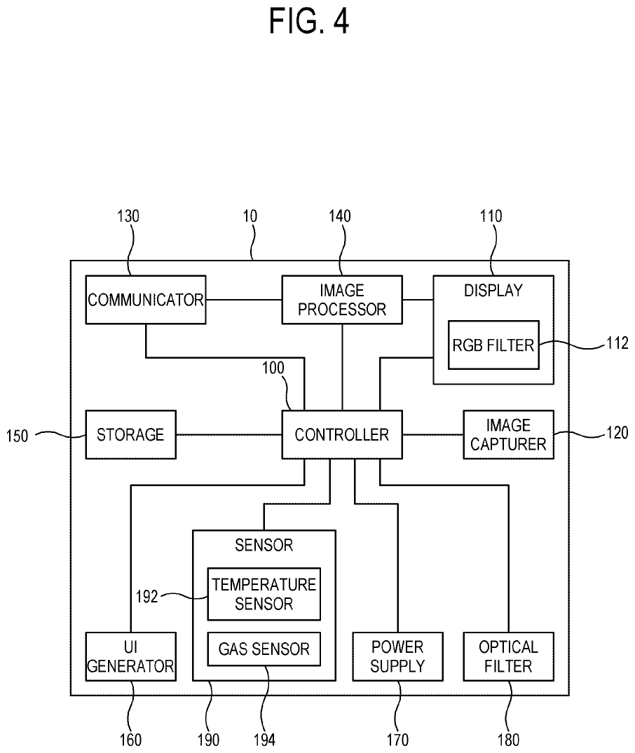

The display 110 may display an image based on an image signal output from an image processor 140 (see FIG. 4). There are no limits to the types of the display 110. There may be various types of the display 110, such as liquid crystal, plasma, light-emitting diode, organic light-emitting diode, surface-conduction electron-emitter, carbon nano-tube, nano-crystal and the like types.

The display 110 may include additional elements in accordance with its types. For example, when the display 110 is of the liquid crystal type, the display 110 may include a liquid crystal display (LCD) panel (not shown), a backlight unit (not shown) for illuminating the LCD panel, and a panel driving board (not shown) for driving the LCD panel.

The display 110 displays an image based on an image signal processed by the image processor 140. There are no limits to a method of displaying an image on the display 110, and the method may include a liquid crystal display (LCD), a plasma display panel (PDP), an organic light emitting diode (OLED), and the like method. In this case, the display may include an LCD panel, a PDP panel, an OLED panel, etc.

The display 110 may display an image and a color calibration process. The display 110 may include a display panel for displaying an image thereon, and a panel driver for processing an input image signal to be displayed as an image on the display panel. However, there are no limits to detailed materialization. An image signal received from an external input source through an interface is subjected to decoding, de-interlacing, scaling and the like image processing processes and then displayed on the display 110. The display 110 has a screen on which an image is displayed, and at least a part of a region of the screen is capable of outputting light having a specific wavelength.

The image capturer 120 is materialized by a camera and captures an external image. The image capturer 120 may capture an image with the second light, i.e. light reflected by projecting the first light, i.e. the blue light output from a partial region of the display 110 on to a body part of a user. When the first light is output, the image capturer 120 may be controlled to capture an image of a user's body in front under control of the controller 100.

The controller 100 may be materialized by a central processing unit (CPU) or a processor, which controls the display 110 to project the first light having a first wavelength range from the first region of the screen on to a user's body, controls the image capturer 120 to capture an image of a user's body, and controls the display 110 to display analysis information about a user's body on the second region of the screen on the basis of the captured image.

FIG. 4 is a block diagram of a user terminal 10 according to another embodiment of the present invention. The user terminal 10 of FIG. 4 according to this embodiment of the present invention may include the display 110 and the image capturer 120 of FIG. 3, and may additionally include a communicator 130, the image processor 140, a storage 150, a UI generator 160, a power supply 170, an optical filter 180, a sensor 190, and the controller 100.

The display 110 includes an RGB filter 112 for outputting the first light. When the controller 100 issues a command to output the first light, power is supplied to the RGB filter 112 of the display 110 so that a partial region of the display 110 can output only the first light having a wavelength of a specific range, i.e. the blue light. Then, when light is emitted from the bottom of the display 110, only the blue light is output from the display 110.

The communicator 130 may receive a signal based on an external input and transmit the signal to the image processor 140 or the controller 100. The communicator 130 may connect with various external input cables to receive a signal from the external input by the cable, or may wirelessly receive a signal in accordance with preset wireless communication standards.

The communicator 130 may include a plurality of connectors (not shown) to which the cables are respectively connected. The communicator 130 may receive a signal from a connected external input, for example, a broadcast signal, an image signal, a data signal and the like based on high definition multimedia interface (HDMI), universal serial bus (USB), and Component standards, or may receive communication data through a communication network.

The communicator 130 may include not only an element for receiving a signal/data from an external input, but also various additional elements such as a wireless communication module (not shown) for wireless communication or a tuner (not shown) for a broadcast signal in accordance with designs of the user terminal 10. The communicator 130 may not only receive a signal from the external device but also transmit the information/data/signal of the user terminal 10 to the external device. That is, the communicator 130 is not limited to only the elements for receiving the signal from the external device, and may be achieved as an interface for interactive communication. The communicator 130 may receive a control signal for selecting a user interface (UI) from a plurality of control devices. The communicator 130 may be materialized by a communication module for publicly known wireless near field communication, such as Bluetooth, Infrared, Ultra-Wideband (UWB), ZigBee, etc., or may be materialized by a publicly known communication port for wired communication. The communicator 130 may be utilized for many purposes of transmitting/receiving data, a command for controlling the display, etc. as well as the control signal for selecting the UI.

The image processor 140 may be materialized by a graphic processing unit (GPU), and there are no limits to the image processing processes performed in the image processor 140. For example, the image processing processes may include de-multiplexing for dividing a predetermined signal into signals according to characteristics, decoding corresponding to an image format of an image signal, de-interlacing for converting an image signal from an interlaced type into a progressive type, noise reduction for improving image quality, detail enhancement, frame refresh rate conversion, etc., and the image processor 140 may include a decoder (not shown) for decoding a source image in accordance with an image format of an encoded source image, and a frame buffer (not shown) for storing the decoded source image in units of frame.

The image processor 140 may be materialized by a system-on-chip (SOC) where such many functions are integrated, or an image processing board (not shown) fabricated by mounting individual elements for performing such processes independently of each other to a printed circuit board. In this manner, the image processor 140 may be built in the display apparatus.

The image processor 140 applies various preset image processing processes to a broadcast signal including an image signal received from a receiver (not shown), and a source image including an image signal received from an image source (not shown). The image processor 140 outputs the image signal subjected to such processes to the user terminal 10, so that the processed source image can be displayed on the user terminal 10.

The storage 150 may be materialized by a writable nonvolatile memory (i.e. read only memory (ROM)) in which data is retained even though the user terminal 10 is powered off and which can reflect a user's changes. That is, the storage 150 may be provided as one of a flash memory, an erasable programmable read only memory (EPROM) and an electrically erasable programmable read only memory (EEPROM). The storage 150 may be configured to store an application program for determining a state of a user's oral cavity. The application program may be stored as a firmware in the storage of the user terminal 10 by a manufacturer, or may be downloaded from an external server when it is not stored in a fabrication stage and installed and executed for determining a state of a user's oral cavity. Alternatively, the application program may be achieved by a web application program to be executed by accessing a server for the web application program, and therefore the application program may be not stored in the storage of the user terminal 10.

The UI generator 160 may generate a UI for operating an application program to be executed. The UI includes a plurality of sub UIs provided in the form of an icon, a text, etc. When a user selects a certain sub UI through the user terminal 10, the application program may operate corresponding to the selected sub UI. That is, each sub UI may be generated in units of a plurality of functions or events for operating the application program running in the user terminal 10.

The UI generator 160 refers to a software or hardware function for generating and controlling a UI displayed on the display 110, and the function may be performed by the controller 100 to be described later. In other words, the UI generator 160 may be configured by a separate chipset, or may be not configured by a separate microprocessor.

The power supply 170 supplies power to internal elements of the user terminal 10, which are in need of the power. Under control of the controller 100, the power supply 170 may supply or cut off power to some elements. Thus, each of the elements starts or stops operating.

The optical filter 180 is provided inside the user terminal 10 and configured to selectively receive incident light traveling from the outside into the image capturer 120. The optical filter 180 may receive power from the power supply 170 and operate to pass the second light of the reflected light but block the light other than the second light. The optical filter 180 may be attached to or slide on the outside of the user terminal 10 so as to cover the outside of the image capturer 120. The optical filter 180 is provided in the form of a film to filter the light of the image capturer 120. In every case of determining the state of the oral cavity, the optical filter 180 may be attached to the image capturer 120 and make it easy to more efficiently determine the second light.

The sensor 190 may include a temperature sensor 192 for optically measuring and sensing a user's body temperature, and a gas sensor 194 for measuring and sensing gas of a bad breath that may be generated in the user's oral cavity. The temperature sensor 192 may be provided as an optical sensor capable of sensing the temperature of a user's oral cavity on the basis of light reflected by projecting the light onto the user's oral cavity, but the temperature sensor 192 is not limited to the optical sensor. The temperature sensor 192 may sense the temperature of a user's oral cavity while or after brushing teeth. Since activation of volatile sulfuric compounds from germs in the oral cavity is closely related with temperature, higher temperature has more effects on reducing the bad breath when a user who has an oral cavity of good conditions brushes teeth. Therefore, it will be more effective to provide coaching, i.e. guide information about teeth-brushing to a user when an optically sensed teeth temperature reaches a temperature having an effect on activities of germs in the oral cavity. The gas sensor 194 may sense gas (e.g. a bad breath), which may be caused by germs in the oral cavity, from a user's expiration. However, when it is determined that a bad breath is generated in a user's oral cavity by a predetermined degree or more, pieces of analysis information based on optical measurements may be combined to determine whether the bad breath is caused after the user consumes food or caused by dental caries, dental plaque and the like in a user's teeth

The controller 100 may control the display 110 to output the first light based on at least one level of the gas and temperature sensed by the sensor 190.

The controller 100 may analyze an image based on the second light having a second wavelength range in the captured image. The controller 100 may analyze at least one among the color, distribution, illumination, fluorescence loss and area of the image based on the second light in the captured image. When a user's oral cavity has a lot of tooth germs, there are many secretions from the tooth germs. Since light having a red fluorescent color is reflected when the first light, i.e. the blue light is projected onto the secretions of the tooth germs, it is possible to determine how many tooth germs are there by analyzing the extent of the light having the red fluorescent color, i.e. at least one of the color, distribution, illumination, fluorescence loss and area of the light. Here, the controller 100 may adjust the first region and the second region on the screen of the display 110 by analyzing the illumination. For instance, as the illumination, i.e. incident angle of the second light to the image capturer 120 is changed, the first region may be increased or decreased and the second region may be increased or decreased.

The controller 100 may set a range of at least one analyzed value to provide analysis information about a user's body. For instance, with respect to the color, the controller 100 may set a pale pink region as an early stage of forming dental plaque. The pale pink region is considered as about 50% in the course of changing from a white green image based on the second light of when there are no tooth germs into red light, and thus the controller 100 may perform setting in this regard. In a case of the fluorescence loss, the controller 100 may set a range of 0% ˜5%, 5%˜20%, and 20% or higher. When the analyzed fluorescence loss has the range of 0%˜5%, 5%˜20%, or 20% or higher, it is possible to determine a degree of dental plaque, and provide information about a diagnosis and teeth care differently according to the ranges.

The controller 100 may extract an image based on the second light from the captured image. By extracting an image having the second light of about 520 nm from the captured image when the optical filter 180 is not mounted or attached, it is possible to determine which region and how much the second light is distributed in the captured image.

The reflected light caused by the first light involves an image of a user's oral cavity, and includes the second light. The second light is of 520 nm and has a fluorescent color tinged with red color dark and light according to its intensity. Therefore, red fluorescent colors may be extracted from the captured image and provided to a user as displayed on the image of the user's oral cavity.

The controller 100 may control the power supply 170 to supply power so that the optical filter 180 can start operating when the first light is output.

The controller 100 may control at least one among the size and position of the first region and the brightness of the first light to be changed corresponding to at least one of the illumination and the brightness of the captured image based on the second light, and the size of the captured image. The controller 100 may determine a suitable size and brightness of the captured image by comparison between the size of the captured image and a preset image size or the size of a previous capture image, thereby adjusting the size and brightness of the first region for outputting the first light. Further, the controller 100 may change the size or position of the first region in accordance with incident angles of the second light to the image capturer 120. For example, when a user uses the user terminal 10 opposite to the user's body as raised much higher than usual, the size of the first region formed in a lower side of the screen of the display 110 becomes smaller and located at a lower position. When a user uses the user terminal 10 opposite to the user's body as raised much lower than usual, the size of the first region becomes larger and thus the controller 100 may decrease the size of the second region. The first region and the second region may have critical values by which the sizes thereof are not decreased and increased any more. Therefore, it is possible to keep the brightness of the first light output from the first region to a minimum. Further, it is possible to keep analysis information about a user's body provided in the second region to a sufficient size. The foregoing examples show that the first region and the second region are variable depending on the position of the user terminal 10.

The controller 100 executes the application program stored in the storage 150 so as to determine a state of a user's oral cavity, and control the UI generator to generate a UI selectable for measuring at least one of a user's dental plaque, dental caries, bad breath and oral cavity temperature.

The analysis information may include at least one among a user's oral state information, oral care information, education and coaching information for the oral cavity, goal setting challenge information, goal attainment guide information, and guide information. The controller 100 may provide a user's oral state information, the oral care information, the education and coaching information for the oral cavity, the goal setting challenge information, the goal attainment guide information, and the guide information together with the captured image, and may provide them to the user through a UI in the form of a table. For example, the oral state information may show progression of dental plaque, and the oral care information may show information about teeth-brushing or management information about the care. The guide information may include the oral state information and the oral care information, and may include information about hospitals or information about dental medicine.

The education and coaching information for the oral cavity refers to information which makes a user be educated on brushing or caring teeth based on the oral state information, and informs a user of a section where brushing is needed for removing dental plaque and bad breath among the teeth, or a detailed caring method such as how to use dental floss. Such education and coaching may be provided using an animation or a character set by a user. With the goal setting challenge and attainment guide information, goal set guide is provided in accordance with a degree of dental plaque or a degree of bad breath, an image is captured again after removing the dental plaque or bad breath by brushing teeth or using dental floss, and the guide about the attainment is converted and provided as a score in accordance with how much the dental plaque or bad breath is removed. By comparison between a measurement result before measuring the dental plaque or bad breath and a measurement result after brushing teeth or using dental floss, higher guide score is set as the dental plaque or bad breath is more removed.

The oral state information, the oral care information, the education and coaching information for the oral cavity, the goal setting challenge information, the goal attainment guide information, and the guide information are not limited to only the foregoing, and may include any related information that can be offered to a user. Such information may be offered having alarm or vibration effect.

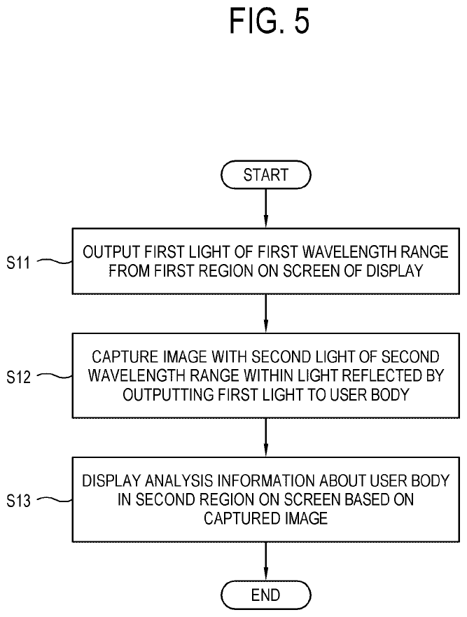

FIG. 5 is a control flowchart of showing a control method of the user terminal 10 according to the present invention.

In the first region on the screen of the display 110, the first light having the first wavelength range is output to a user's body (S11).

The image capturer 120 captures an image of the user's body under control of the controller 100 (S12).

Based on the captured image, the analysis information about the user's body is displayed on the second region on the screen (S13).

The first light in the operation S11 may be output from not the display apparatus 10 but an external device, and the control flow may be performed by receiving an image captured with regard to a region to which the first light having the first wavelength range is output. The control flow is merely one embodiment, and there are no limits to this embodiment.

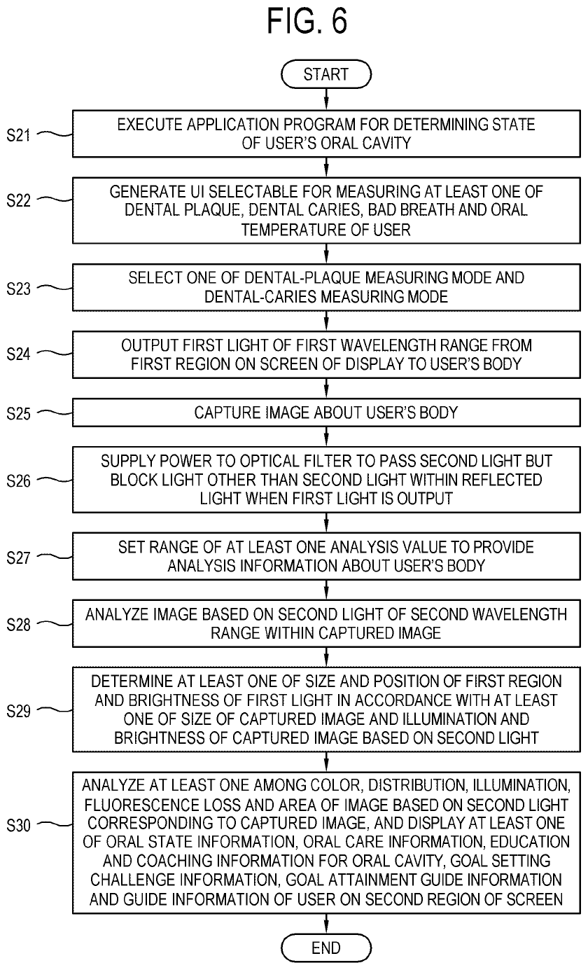

FIG. 6 is a control flowchart of showing a control method of the user terminal 10 according to one embodiment of the present invention.

A user selects and executes an application program for determining a state of a user's oral cavity from the screen of the display 110 (S21).

The controller 100 control the UI generator 160 to generate a UI selectable for measuring at least one among the dental plaque, dental caries, bad breath, and oral cavity temperature of the user (S22).

The user selects one of a generated dental-plaque measuring mode UI and a generated dental-caries measuring mode UI (S23).

The controller 100 controls the display 110 so that the first region on the screen of the display 110 can output the first light having a first wavelength range to the user's body in accordance with the selected mode (S24).

The image capturer 120 captures an image with regard to the user's body (S25).

The power supply 170 supplies power to the optical filter 180 to pass the second light of the reflected light but blocks the light other than the second light when the first light is output (S26).

To provide analysis information about the user's body, a range of at least one analysis value is set (S27). The range of the analysis value maybe previously set by a user or with reference to a doctor's advice.

The image based on the second light having a second wavelength range is analyzed within the captured image (S28).

The controller 100 determines at least one of the size and position of the first region and the brightness of the first light in accordance with at least one of the size of the captured image and the illumination and brightness of the captured image based on the second light (S29). The controller 100 controls the display 110 to have the determined size and positions of the first region and the determined brightness of the first light.

The controller 100 analyzes at least one among the color, distribution, illumination, fluorescence loss and area of the image based on the second light corresponding to the captured image, and displays at least one of the oral state information, oral care information, education and coaching information for the oral cavity, goal setting challenge information, goal attainment guide information and guide information of the user on the second region of the screen (S30). For instance, the image of the user's oral cavity may be displayed with colors to show a degree of bad breath, dental caries or dental plaque; the analysis information may be represented with digits; vibration or alarm may be given in accordance with the degrees of dental caries, dental plaque and bad breath; and the image based on the colors may be displayed with a table in which the analysis information is tabulated with digits.

The first light may be output from not the display apparatus 10 but an external device, and the control flow may be performed by receiving an image captured with regard to a region to which the first light having the first wavelength range is output. The control flow is merely one embodiment, and there are no limits to this embodiment.

In addition to the control flow, the controller 100 may measure a gas level in the user's oral cavity and measure a temperature level when the gas level is equal to or higher than a predetermined level. The controller 100 may measure the temperature level in the user's oral cavity and capture an image by outputting the first light when the temperature level is lower than or equal to a predetermined level. When the gas level in the user's oral cavity is lower than or equal to a predetermined level, the controller 100 may control the guide information to be displayed for guiding the measured gas level, i.e. a degree of bad breath without outputting the first light and capturing the image. When the temperature level in the user's oral cavity is equal to or higher than a predetermined level, the controller 100 may control the guide information to be displayed for guiding the measured temperature level and a bad breath level without outputting the first light and capturing the image.

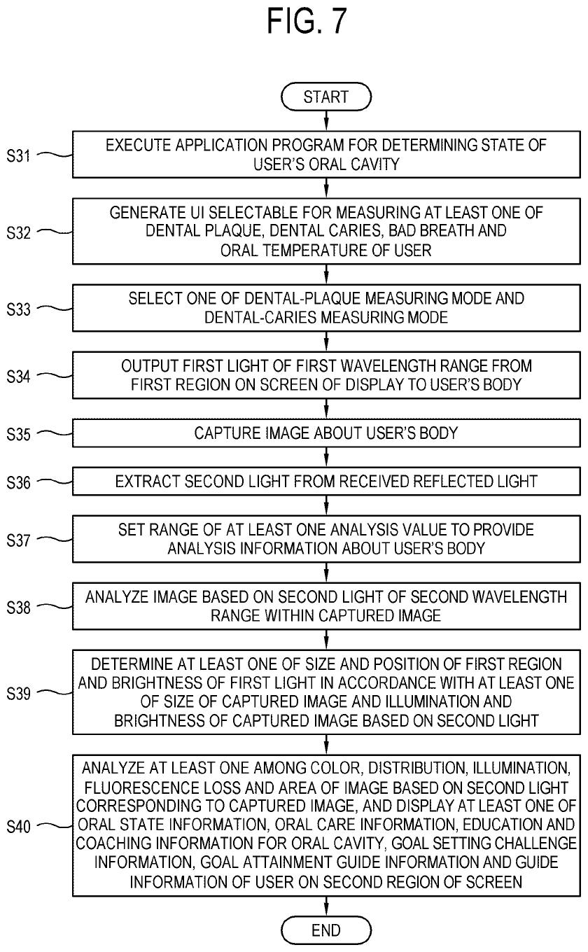

FIG. 7 is a control flowchart of showing a control method of a user terminal according to another embodiment of the present invention.

A user selects and executes an application program for determining a state of a user's oral cavity from the screen of the display 110 (S31).

The controller 100 control the UI generator 160 to generate a UI selectable for measuring at least one among the dental plaque, dental caries, bad breath, and oral cavity temperature of the user (S32).

The user selects one of a generated dental-plaque measuring mode UI and a generated dental-caries measuring mode UI (S33).

The controller 100 controls the display 110 so that the first region on the screen of the display 110 can output the first light having a first wavelength range to the user's body in accordance with the selected mode (S34).

The image capturer 120 captures an image with regard to the user's body (S35).

The controller 100 extracts only the second light from the received reflected light (S36). To extract only the wavelength range of the second light, an algorithm may be used to determine which region and how much the second light of the received reflected light is distributed in the captured image.

To provide analysis information about the user's body, a range of at least one analysis value is set (S37). The range of the analysis value maybe previously set by a user or with reference to a doctor's advice.

The image based on the second light having a second wavelength range is analyzed within the captured image (S38).

The controller 100 determines at least one of the size and position of the first region and the brightness of the first light in accordance with at least one of the size of the captured image and the illumination and brightness of the captured image based on the second light (S39). The controller 100 controls the display 110 to have the determined size and positions of the first region and the determined brightness of the first light.

The controller 100 analyzes at least one among the color, distribution, illumination, fluorescence loss and area of the image based on the second light corresponding to the captured image, and displays at least one of the oral state information, oral care information, education and coaching information for the oral cavity, goal setting challenge information, goal attainment guide information and guide information of the user on the second region of the screen (S40). For instance, the image of the user's oral cavity may be displayed with colors to show a degree of bad breath, dental caries or dental plaque; the analysis information may be represented with digits; and the image based on the colors may be displayed with a table in which the analysis information is tabulated with digits.

The first light may be output from not the display apparatus 10 but an external device, and the control flow may be performed by receiving an image captured with regard to a region to which the first light having the first wavelength range is output. The control flow is merely one embodiment, and there are no limits to this embodiment.

In addition to the control flow, the controller 100 may measure a gas level in the user's oral cavity and measure a temperature level when the gas level is equal to or higher than a predetermined level. The controller 100 may measure the temperature level in the user's oral cavity and capture an image by outputting the first light when the temperature level is lower than or equal to a predetermined level. When the gas level in the user's oral cavity is lower than or equal to a predetermined level, the controller 100 may control the guide information to be displayed for guiding the measured gas level, i.e. a degree of bad breath without outputting the first light and capturing the image. When the temperature level in the user's oral cavity is equal to or higher than a predetermined level, the controller 100 may control the guide information to be displayed for guiding the measured temperature level and a bad breath level without outputting the first light and capturing the image.

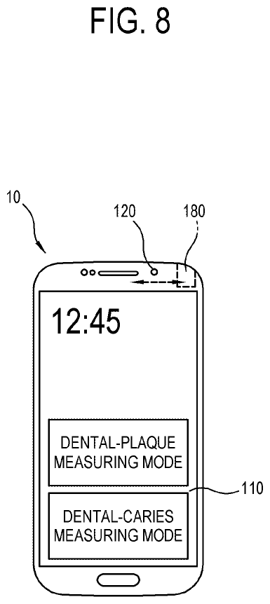

FIG. 8 illustrates an example of showing a user interface (UI) for selecting a user's dental plaque and dental caries in a user terminal according to another embodiment of the present invention,

When a user selects the application program UI for determining the user's oral cavity, the dental-plaque measuring mode and the dental-caries measuring mode may be displayed. The user may select one of the displayed dental-plaque measuring mode and the displayed dental-caries measuring mode to enter the measuring mode. Besides, other measuring modes may be displayed, or the dental-caries measuring mode may be directly implemented.

In an upper side of the user terminal 10, the image capturer 120 is provided. At the right side of the image capturer 120, the optical filter 180 may be provided to cover the lens of the image capturer. The optical filter 180 may slide leftward and rightward to cover the image capturer 120. Further, the optical filter 180 may be detachably provided and attached to a lens portion of the image capturer 120.

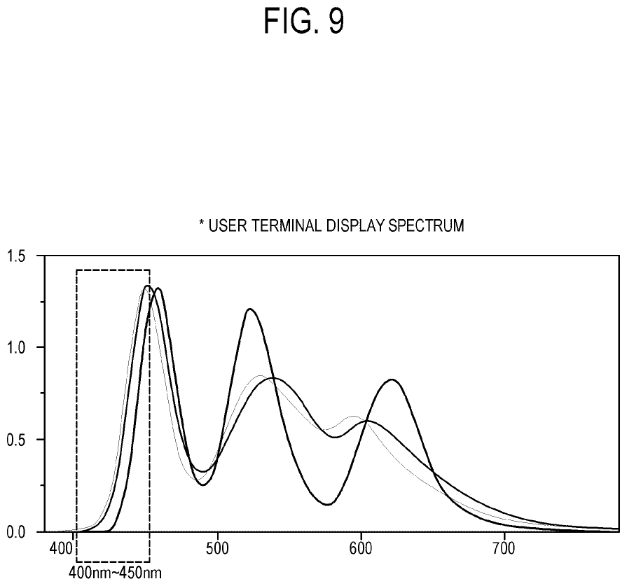

FIG. 9 is a graph of showing a screen spectrum of outputting blue light in a user terminal according to one embodiment of the present invention.

This shows that various user terminals 10 outputs light having a range of 400-450 nm, i.e. blue light. Likewise, the light may be output having a wavelength of a different range.

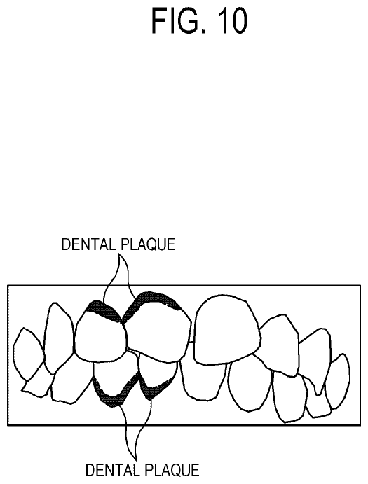

FIG. 10 illustrates an example of showing analysis information about a user body on a captured image in the user terminal 10 according to one embodiment of the present invention.

This shows that the user terminal 10 captures the image with the second light received after outputting the first light, and displays the captured image with analysis information. Specifically, the image of the user's oral cavity is captured and displayed, in which the light reflected by outputting the first light to the oral cavity involves the second light having red fluorescence caused by the secretions of the tooth germs, and a portion marked with this red fluorescent color indicates the dental plaque. In accordance with extent of the portion marked with the red fluorescent color, i.e. the extent of the color, distribution, illumination, and fluorescence loss and area, it is possible to determine a degree of dental caries, a degree of dental plaque, a degree of accumulated plaque, a degree of bad breath, etc.

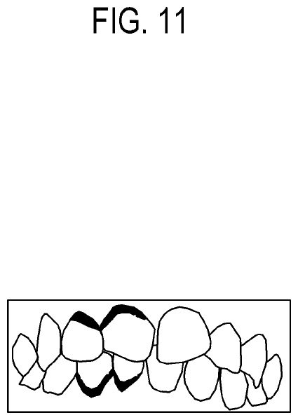

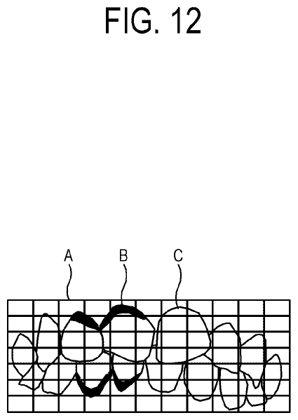

FIGS. 11 and 12 illustrate examples of analyzing a color and an area through a grid region set on a captured image in a user terminal 10 according to one embodiment of the present invention.

In FIGS. 11 and 12, the color and area of the portion marked with the red fluorescence of FIG. 10 are analyzed by setting the grid region.

White/red colors are detected, and a number ratio of white/red grids A is calculated, thereby determining a degree of dental caries/dental plaque. As the grid A is more densely given, an accuracy of measuring the dental caries/dental plaque B in a user's teeth C will become higher. One grid is regarded as a cell for determining the dental caries/dental plaque, and it is determined whether the grid is red, white-green or white. When the grid is red, it is determined as the dental caries/dental plaque. When the grid is white-green or white, it is determined as normal.

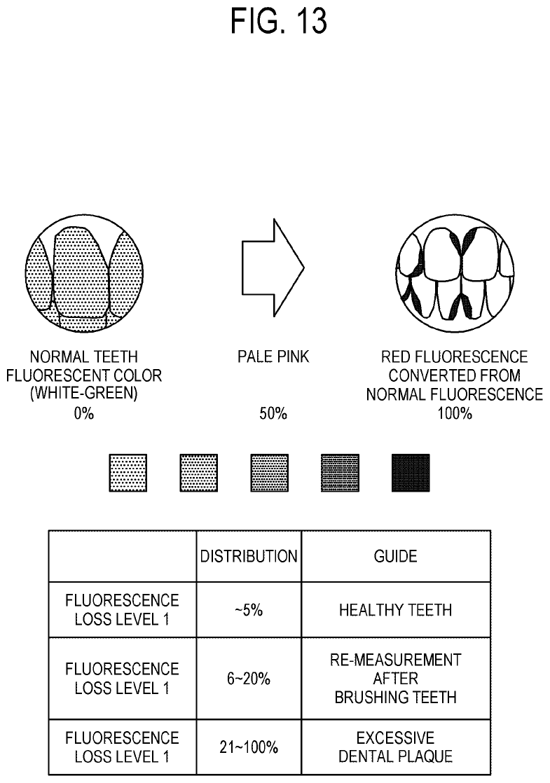

FIG. 13 illustrates an example of providing state information and diagnostic guide information for determining a state of teeth state on a captured image in a user terminal 10 according to one embodiment of the present invention.

When the blue light is output from the first region of the user terminal 10 to a user's oral cavity in which the user has healthy teeth without dental caries or dental plaque, the image captured by the image capturer 120 shows normal teeth fluorescent color (white-green) with red fluorescence of 0% as shown therein. On the other hand, when the user's teeth has the dental caries or dental plaque, for example, when the red fluorescence is of about 50%, i.e. pale pink, it shows that conversion of the dental caries or dental plaque on the teeth into the tartar is progressed by about 50%., the red fluorescence is converted from the normal fluorescence by 100%, it shows that the dental plaque is progressed significantly.

A percentage (%) is expressed by comparison between the whole number of grids and the number of grids corresponding to the dental caries/dental plaque. When total grids are 100 and red grids corresponding to the dental caries/dental plaque are 10, it is determined as 10%. When one grid half red and half white, one grid may be determined as the dental caries/dental plaque, or may be also expressed as a percentage (%) by dividing it into smaller parts. In the foregoing example, when five grids has half red and half white among ten grids corresponding the dental caries/dental plaque, ten grids are determined as the dental caries/dental plaque, or the total number of grids is increased from one hundred to one thousand by further dividing the grid into smaller parts to thereby show a percentage (%) of the dental caries/dental plaque.

Referring to FIG. 13, color indexes are given below with regard to pictures changed in color, and a table (reference) is given below the color indexes to show distribution and guide with regard to fluorescence loss levels. When the fluorescence loss level is lower than or equal to 5%, the state information and diagnostic guide information are provided to inform that teeth are healthy. Regarding the distribution of 6-20%, the state information and the diagnostic guide information are provided to advise trying measurement again after brushing the teeth.

Regarding the distribution of 21-100%, the table informs that the dental plaque is serious. The values of the distribution percentage (%) may be varied depending on medical determination references for diagnostic and dental care information. The range is merely an example, and the guide may be differently given by setting the percentage of the distribution according to embodiments and various intentions.

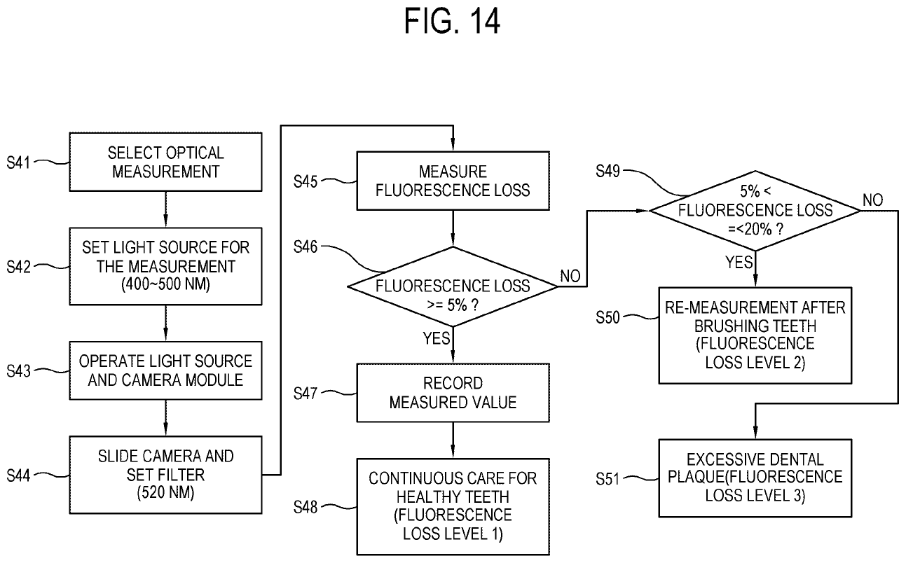

FIG. 14 is a flowchart of showing a control flow for measuring a fluorescence loss and providing analysis information about dental plaque in the user terminal 10 according to one embodiment of the present invention. However, the range is merely an example, and the guide may be differently given by setting the percentage of the distribution according to embodiments and various intentions.

An application program is selected to optically measure a state of a user's teeth (S41).

A light source for the measurement is set (S42). The light source for the measurement may be previously set to 400-500 nm.

The first light is output from the first region to a user's oral cavity, and a camera module, i.e. the image capturer operates (S43).

The optical filter 180 slides and positions to cover the lens of the image capturer 120 (S44). The optical filter 180 may be provided as a built-in element and operate by receiving power. Alternatively, the optical filter 180 may be detachably provided and configured to cover the image capturer 120.

The fluorescence loss is measured in the image captured with the second light (S45).

When the measured fluorescence loss is lower than 5% (S46), the measured value is stored in the storage 150 (S47).

The second region displays information for guiding that the fluorescence loss of the second light is lower than 5% and teeth are healthy with reference to the distribution and guide of FIG. 13 (S48). To continuously take care of a user's teeth, the records stored in the storage 150 may be used.

It is determined whether the measured fluorescence loss is within a range of 5% to 20% (S49).

When the measured fluorescence loss is within the range of 5˜20%, the second region may display a guide message for informing that the fluorescence loss is within the range of 5˜20% and advising trying measurement again after brushing teeth (S50). When the fluorescence loss is measured again, oral education and coaching information about brushing teeth and using dental floss for the oral cavity may be offered. The oral education information refers to information that makes a user be educated on a teeth brushing method or a teeth care method based on the distribution percentage and the guide of FIG. 13, and informs a brushing section, from which the dental plaque and bad breath have to be removed, of the teeth or a detailed method for dental care such as a method of using the dental floss, etc. Such education and coaching information may be provided an animation or a character set by a user. The range is merely an example, and the guide phrase may be differently displayed by setting the percentage of the fluorescence loss according to embodiments or various intentions.

When the measured fluorescence loss is greater than 20%, the second region may display information for guiding that the florescence loss is greater than 20% and the dental plaque is currently excessive (S51). Further, a guide message may be displayed to advise a user to visit a hospital and receive dental treatment since the dental caries are serious. Further, information about dental clinics around a current location, a house, an office, etc. may be guided. Further, information about brushing teeth for removing the measured excessive dental plaque may be additionally guided.

The first light may be output from not the display apparatus 10 but an external device, and the control flow may be performed by receiving an image captured with regard to a region to which the first light having the first wavelength range is output. The control flow is merely one embodiment, and there are no limits to this embodiment.

In addition to the control flow, the controller 100 may measure a gas level in the user's oral cavity and measure a temperature level when the gas level is equal to or higher than a predetermined level. The controller 100 may measure the temperature level in the user's oral cavity and capture an image by outputting the first light when the temperature level is lower than or equal to a predetermined level. When the gas level in the user's oral cavity is lower than or equal to a predetermined level, the controller 100 may control the guide information to be displayed for guiding the measured gas level, i.e. a degree of bad breath without outputting the first light and capturing the image. When the temperature level in the user's oral cavity is equal to or higher than a predetermined level, the controller 100 may control the guide information to be displayed for guiding the measured temperature level without outputting the first light and capturing the image.

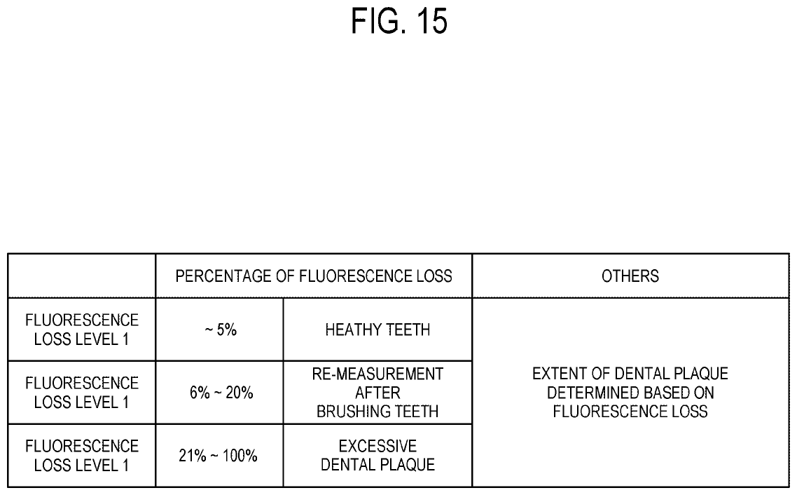

FIG. 15 illustrates an example of the analysis information obtained from the control flow of FIG. 14,

In FIG. 15, information including at least one of the oral state information, the oral care information, the education and coaching information for the oral cavity, the goal setting challenge information, the goal attainment guide information and the guide information from the control flow of FIG. 14 is tabulated. With respect to the fluorescence loss, it is possible to determine a degree of dental plaque.

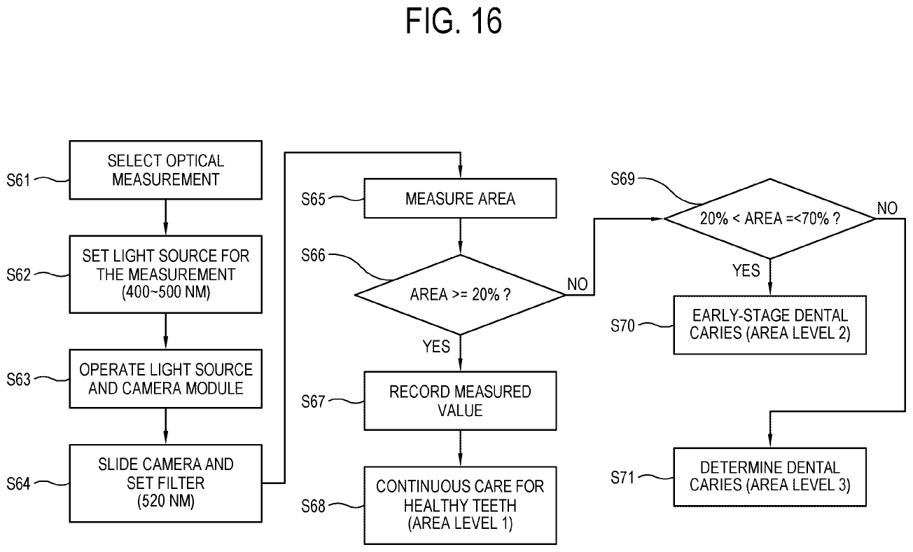

FIG. 16 is a flowchart of showing a control flow for measuring an area of second light and providing analysis information about dental caries in the user terminal 10 according to another embodiment of the present invention.

An application program for optically measuring a state of a user's teeth is selected (S61).

A measurement light source is set (S62). The measurement light source may be previously set to 400˜500 nm.

The first light is output from the first region to the user's oral cavity, and a camera module, i.e. the image capturer operates (S63).

The optical filter 180 slides to cover the lens of the image capturer 120 (S64). The optical filter 180 may be provided as a built-in element and operate by receiving power. Alternatively, the optical filter 180 may be detachably provided and configured to cover the image capturer 120.

The area of the second light in the captured image is measured (S65).

When the measured area of the second light is smaller than 20% (S66), a measured value is stored in the storage 150 (S67).

The second region displays information for guiding that the area of the second light is smaller than 20% and teeth are healthy (S68). To continuously take care of a user's teeth, the records stored in the storage 150 may be used.

It is determined whether the measured area of the second light is within a range of 20˜70% (S69).

When the measured area of the second light is within the range of 20˜70%, the second region may display a guide message for informing that the area of the second light is within the range of 20˜70% and the state of dental caries is determined as an early stage (S70). However, the foregoing range is merely an example, and the guide message may be displayed by setting the fluorescence loss to another range according to another embodiment or various intentions.

When the measured area of the second light is greater than 70%, the second region may display information for guiding that the area of the second light is greater than 20% and the teeth have the dental caries (S71). Further, a guide message may be displayed to advise a user to visit a hospital and receive dental treatment since the dental caries are serious. Further, information about dental clinics around a current location, a house, an office, etc. may be guided. Further, information about brushing teeth for removing the measured excessive dental plaque may be additionally guided. The information about brushing the teeth may be the education and coaching information for the oral cavity with regard to brushing the teeth and using the dental floss.

The education information for the oral cavity refers to information that makes a user be educated on a teeth brushing method or a teeth care method based on the distribution percentage and the guide of FIG. 13, and informs a brushing section, from which the dental plaque and bad breath have to be removed, of the teeth or a detailed method for dental care such as a method of using the dental floss, etc. Such education and coaching information may be provided an animation or a character set by a user.

In addition, the dental plaque may be formed subsequent to the dental caries, and thus a guide message may be displayed to advise the measurement of the dental plaque.

The first light may be output from not the display apparatus 10 but an external device, and the control flow may be performed by receiving an image captured with regard to a region to which the first light having the first wavelength range is output. The control flow is merely one embodiment, and there are no limits to this embodiment.

In addition to the control flow, the controller 100 may measure a gas level in the user's oral cavity and measure a temperature level when the gas level is equal to or higher than a predetermined level. The controller 100 may measure the temperature level in the user's oral cavity and capture an image by outputting the first light when the temperature level is lower than or equal to a predetermined level. When the gas level in the user's oral cavity is lower than or equal to a predetermined level, the controller 100 may control the guide information to be displayed for guiding the measured gas level, i.e. a degree of bad breath without outputting the first light and capturing the image. When the temperature level in the user's oral cavity is equal to or higher than a predetermined level, the controller 100 may control the guide information to be displayed for guiding the measured temperature level without outputting the first light and capturing the image.

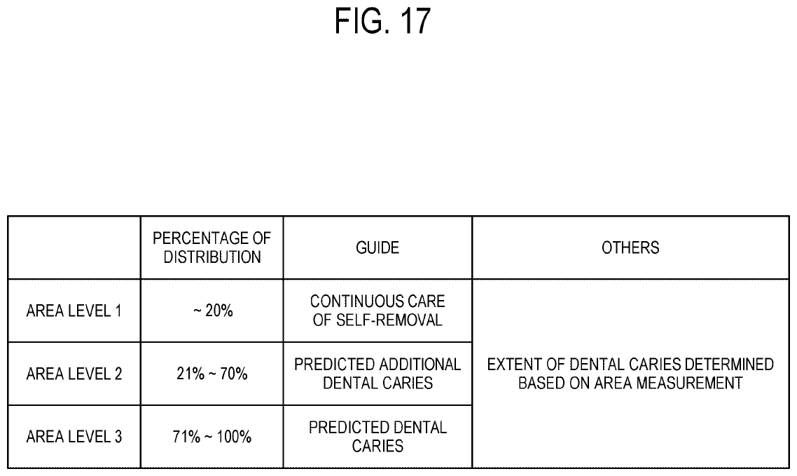

FIG. 17 illustrates an example of the analysis information obtained from the control flow of FIG. 16.

In FIG. 17, information including at least one of the oral state information, the oral care information, the education and coaching information for the oral cavity, the goal setting challenge information, the goal attainment guide information and the guide information from the control flow of FIG. 16 is tabulated. With respect to the area of the second light, it is possible to determine a degree of dental plaque. When the area of the second light has extent of 0˜20%, a guide message for continuous care of self-removal is displayed on the second region. When the area of the second light has extent of 21˜70%, a guide message for prediction of early-stage dental caries is displayed on the second region. When the area of the second light has extent of 70˜100%, a guide message for prediction of the dental caries is displayed on the second region.

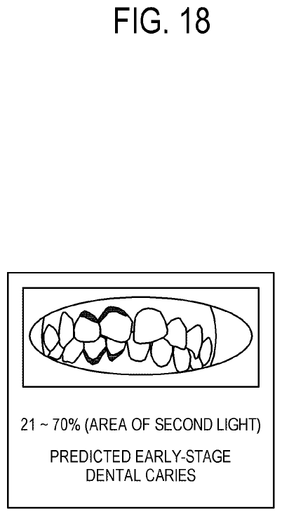

FIG. 18 illustrates an example of displaying guide information on the second region in the user terminal 10 according to one embodiment of the present invention.

In FIG. 17, when the area of the second light has extent of 21˜70%, the second region may display “21˜70% (the area of the second light), and the early-stage dental caries is predicted”. Further, phrases such as “risk for dental caries”, “please visit a hospital”, “dental clinics around the current location—OO hospital, ** hospital”, “teeth-brushing information—attached” and “dental-plaque measuring mode execution”, etc. may be displayed. When such a phrase is displayed, it may be provided using an animation or a character set by a user.

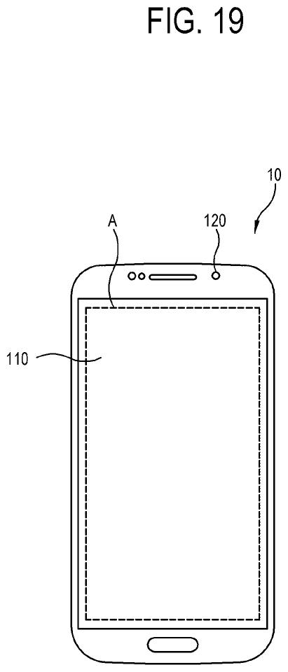

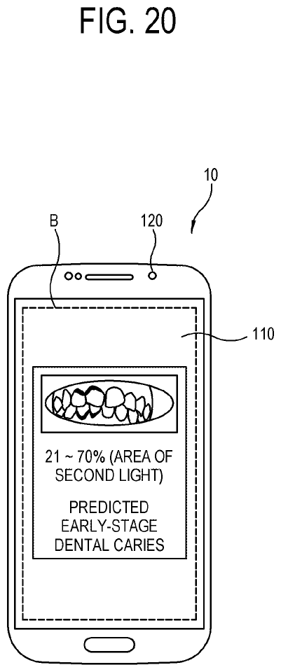

FIGS. 19 and 20 illustrate examples of showing the first region and the second region on the screen of the display 110 in the user terminal 10 according to still another embodiment of the present invention.

In the user terminal 10 according to the present invention, the image is captured by outputting the first light from the first region on the screen of the display 110, and the captured image is analyzed so that the analysis information can be displayed in the second region on the screen of the display 110.

FIG. 19 shows that the first light is output from the whole region on the screen of the display 110. In this case, the first region is the whole region on the screen of the display 110. FIG. 20 shows that the first region stops outputting the first light and then the analysis information is displayed in the whole region on the screen of the display 110. In this case, the first region and the second region alternately outputs the first light and display displays the captured image through one whole region on the screen of the display 110, and the first region and the second region may overlap with each other.

According to one embodiment, to get more detailed analysis information in the state that the first region is outputting the first light and the second region is displaying the analysis information, the first region may extend across the whole region on the screen of the display 110 while making the second region disappear. To display the analysis information largely, the second region may extend across the whole region on the screen of the display 110 while making the first region disappear. When each of the first region and the second region extends and disappears, the region may gradually extend or disappear, or may extend or disappear at a time. The first region and the second region may overlap with each other or may not overlap with each other.

According to another embodiment, the user terminal 10 may have an automatic rotation function. When the user terminal 10 is rotated in the state that the first region is outputting the first light and the second region is displaying the analysis information, the areas of the first region and the second region may be changed corresponding to the rotation. For instance, when the first region before the rotation occupies 40% rectangular area of the whole screen left and right width from the bottom, the first region may be changed to occupy 40% rectangular area of the whole screen left and right width from the bottom even after the user terminal is rotated. However, when the screen of the display 110 in the user terminal 10 is rotated by 90 degrees and changes from a portrait orientation to a landscape orientation in the state that the first region is outputting the first light and the second region is displaying the analysis information, the first region and the second region may also change in area corresponding to the rotation. However, the areas of the first region and the second region are changed corresponding to the rotation, each region may be stretched long in left and right directions or up and down directions. To avoid this, each region may not change even though the user terminal 10 rotates. Further, to make a user easily read the analysis information, only the second region for the analysis information may change corresponding to the rotation of the user terminal 10.

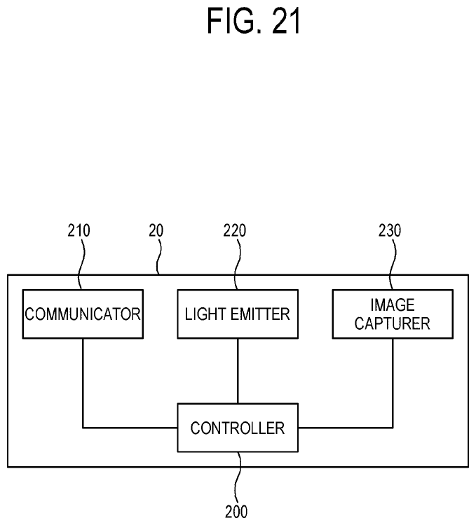

FIG. 21 is a block diagram of an oral care apparatus according to the present invention. The oral care apparatus 20 according to the present invention may include a communicator 210, a light emitter 220, an image capturer 230 and a controller 200.

The communicator 210 may receive a signal based on an external input and transit the received signal to the controller 200. The communicator 210 may receive a signal from an external input by connection of various external input cables, or may wirelessly receive a signal in accordance with preset communication standards.

The communicator 210 may include a plurality of connectors (not shown) to which the cables are connected. The communicator 210 may receive a signal from a connected external input, for example, a broadcast signal, an image signal, a data signal, etc. according to high definition multimedia interface (HDMI), universal serial bus (USB) and component standards, or may receive communication data through a communication network.

The communicator 210 may include not only elements for receiving a signal/data from an external input, but also various additional elements such as a wireless communication module (not shown) for wireless communication, a tuner (not shown) for receiving a broadcast signal, etc. in accordance with design of the oral care apparatus 20. Besides receiving a signal from an external device such as the user terminal, the communicator 210 may transmit information/data/a signal from the oral care apparatus 20 to the external device. That is, the communicator 210 is not limited to the elements for receiving a signal from an external device, and may be materialized by an interface for interactive communication. The communicator 210 may receive a control signal for selecting a UI from a plurality of control devices. The communicator 130 may be materialized by communication modules for publicly known near field communications such as Bluetooth, Infrared (IR), ultra wideband (UWB), ZigBee, etc., and may be materialized by publicly known communication ports for the wired communication.

The light emitter 220 may be configured to emit the first light having the first wavelength range, i.e. blue light along with an RGB filter provided on the display screen. The first light may have a wavelength of 400-450 nm.

The image capturer 230 may be materialized by a camera to capture an image of the outside. The image capturer 230 may capture an image with the second light, i.e. light reflected from a user's body part as the light emitter 220 outputs the first light, i.e. the blue light. Under control of the controller 200, the image capturer 230 is controlled to capture an image of a user's body in front, in particular, a user's oral cavity when the first light is output.

The controller 200 may control the light emitter 220 to output the first light having the first wavelength range, control the image capturer 230 to capture an image, and control the communicator 210 to transmit the captured image to the user terminal 10.

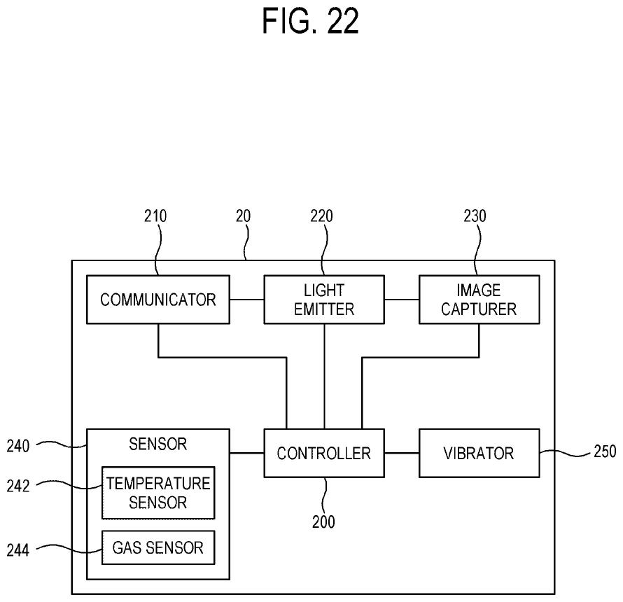

FIG. 22 is a block diagram of the oral care apparatus according to one embodiment of the present invention. According to one embodiment of the present invention, the oral care apparatus 20 may include the elements shown in FIG. 21 and further include a sensor 240 and a vibrator 250.

The sensor 240 may include a temperature sensor 242 for optically measuring and sensing a user's body temperature, and a gas sensor 244 for measuring and sensing gas of bad breath that may occur in a user's oral cavity. The temperature sensor 242 may be materialized by an optical sensor capable of sensing a temperature of a user's oral cavity based on light reflected and received by emitting light to a user's oral cavity, but not limited thereto. The temperature sensor 242 may measure a body temperature of a user's oral cavity when or after brushing teeth. Since activation of volatile sulfuric compounds from germs in the oral cavity is closely related with temperature, higher temperature has more effects on reducing the bad breath when a user who has an oral cavity of good conditions brushes teeth. Therefore, it will be more effective to provide coaching, i.e. guide information about teeth-brushing to a user when an optically sensed teeth temperature reaches a temperature having an effect on activities of germs in the oral cavity. The gas sensor 194 may sense gas (e.g. a bad breath), which may be caused by germs in the oral cavity, from a user's expiration. However, when it is determined that a bad breath is generated in a user's oral cavity by a predetermined degree or more, pieces of analysis information based on optical measurements may be combined to determine whether the bad breath is caused after the user consumes food or caused by dental caries, dental plaque and the like in a user's teeth.

The vibrator 250 may make the oral care apparatus 20 oscillate under control of the controller 200. For instance, when a user's body temperature does not rise up to a predetermined level or higher while brushing teeth using the oral care apparatus 20, the controller 200 may control the vibrator 250 to operate so that the user's body temperature can rise up to the predetermined level or higher. When a control signal or the like signal is received from the user terminal 10 or the like external device, the vibrator 250 may generate vibration for informing that the signal is received.

The controller 200 may control the communicator 210 to transmit at least one of a gas level and a temperature level sensed by the sensor 240 to the user terminal 10. The controller 200 may receive a control signal, which commands measurement of a user's bad breath, from the user terminal 10 or the like external device. In this case, the controller 200 may control the vibrator 250 to operate and inform a user of that. When a user inputs expiratory breath to the sensor 240 of the oral care apparatus 20 in response to the information given by the vibrator 250, the gas sensor 244 measures and senses the gas level. The controller 200 may control the temperature sensor 242 to sense the temperature level when the gas level sensed by the gas sensor 244 is equal to or higher than a preset level. On the other hand, when the sensed gas level is lower than the preset level, the controller 200 may determine that the teeth are healthy, and send the sensed gas level to the user terminal 10 or the like external device so that a user can be informed of that the teeth are healthy. When the temperature level sensed by the temperature sensor 242 is lower than or equal to a present temperature level, the controller 200 may control the light emitter 220 to output the first light to a user's oral cavity. When the temperature level is higher than the preset temperature level, the controller 200 transmits the sensed temperature level to the user terminal 10 or the like external device to thereby inform a user of that. The controller 200 may perform control to capture an image of a part to which the first light is output, and transmit the captured image to the user terminal 10 or the like external device.

The controller 200 may perform control to perform at least one of sensing at least one of the gas and the temperature in a user's oral cavity, and outputting the first light, based on the control signal received from the user terminal 10.

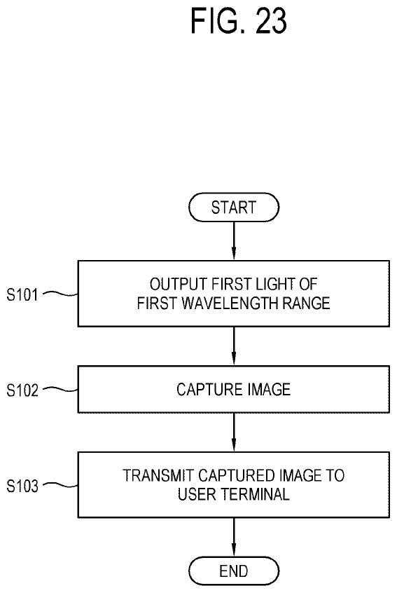

FIG. 23 is a flowchart of showing operations of the oral care apparatus 20 according to the present invention.

The first light of the first wavelength range is output (S101). An image is captured (S102). The captured image is transmitted to the user terminal 10 or the like external device (S103).

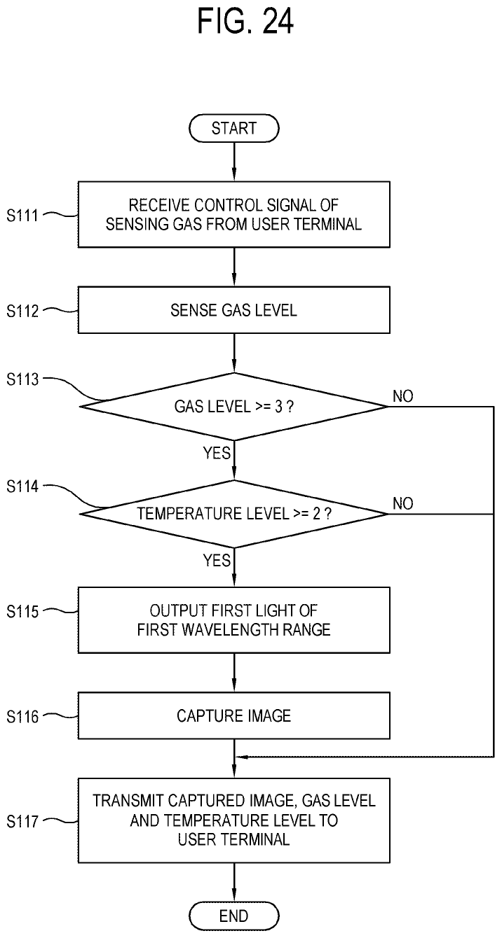

FIG. 24 is a flowchart of showing operations of the oral care apparatus 20 according to one embodiment of the present invention.

A control signal issued for sensing bad breath gas in a user's oral cavity is received from the user terminal 10 (S111).

A bad breath gas level is sensed (S112).

It is determined whether the bad breath gas level is equal to or higher than a preset bad breath gas level of ‘3’ (S113).

When the sensed bad breath gas level is equal to or higher than the preset bad breath gas level of ‘3’, a temperature level in a user's oral cavity is sensed (S114).

When the sensed temperature level is lower than or equal to a preset level of ‘2’, the first light of the first wavelength range is output (S115).

An image is captured (S116).

When the sensed bad breath gas level is lower than the preset bad breath gas level of ‘3’ or the sensed temperature level is higher than the preset level of ‘2’, the sensed bad breath gas level and the sensed temperature level are transmitted to the user terminal 10, and the captured image is also transmitted to the user terminal 10 (S117).

The controller 200 performs the operations by sensing the bad breath gas level and the temperature level and comparing them with the preset levels. Alternatively, the operations may be performed by transmitting the sensed bad breath gas level and temperature level to the user terminal 10 and receiving the control signal.

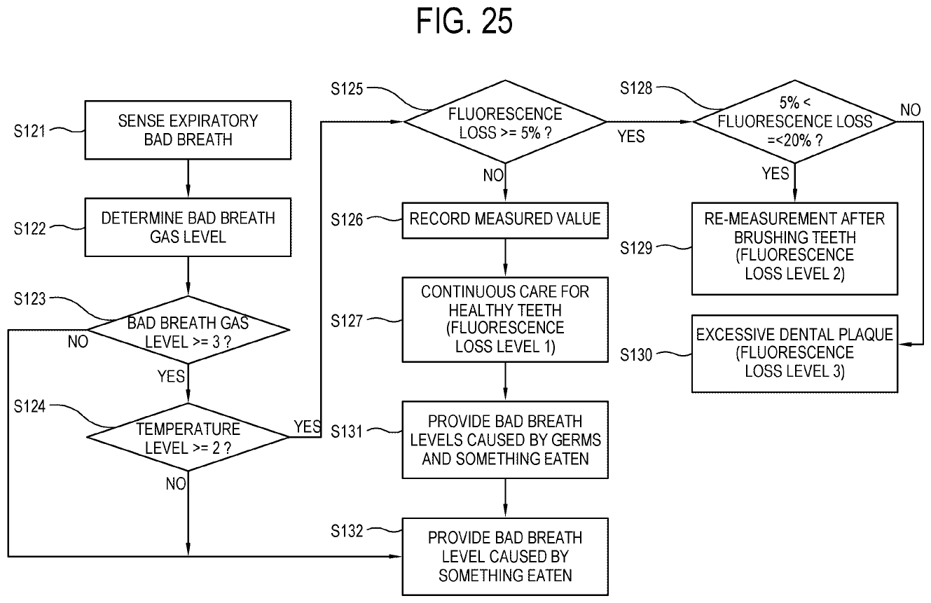

FIG. 25 is a flowchart of showing a control flow of providing analysis information based on measurement of a user's bad breath, oral temperature and light in the oral care apparatus 20 and the user terminal 10 according to one embodiment of the present invention.

As a user breathes out his/her breath toward the gas sensor 244 of the oral care apparatus 20, the user's bad breath gas is sensed to obtain a bad breath gas level (S121).

The gas sensor 244 transmits the sensed bad breath gas level to the controller 200 (S122).

The controller 200 determines whether the sensed bad breath gas level is equal to or higher than the preset bad breath gas level of ‘3’ (S123).

When the sensed bad breath gas level is equal to or higher than the preset bad breath gas level of ‘3’, a temperature level in the user's oral cavity is sensed (S124).

When the sensed temperature level is equal to or higher than the preset level of ‘2’, the first light of the first wavelength range is output toward the user's oral cavity. An image of the user's oral cavity is captured. Fluorescence loss in the captured image is determined (S125).

When the fluorescence loss of the captured image is equal to or lower than 5%, measured values are stored (S126).

The information about the fluorescence loss equal to or lower than 5% and the healthy teeth is displayed (S127). With the stored records, it is possible to continuously take care of the user's teeth.

It is determined whether the fluorescence loss of the captured image is within a range of 5% to 20% (S128).

When the measured fluorescence loss is within the range of 5-20%, a guide message may be displayed to inform that the fluorescence loss of the captured image is within a range of 5% to 20% and to advise trying the measurement again after brushing teeth may be displayed (S129). However, the foregoing range is merely an example, and the fluorescence loss of another range may be set to display a guide phrase in accordance with other embodiments or various intentions.

When the measured fluorescence loss is greater than 20%, it may be informed that the fluorescence loss of the captured image is greater than 20% and the current dental plaque is in an excessive state (S130). Further, a guide message may be displayed to advise visiting a hospital and receiving dental treatment since the dental caries are serious. Further, information about dental clinics around a current location, a house, an office, etc. may be guided. Further, information about brushing teeth for removing the measured excessive dental plaque may be additionally guided.

When the sensed gas level is lower than the preset level of ‘3’ or the sensed temperature level is lower than the preset level of ‘2’, the sensed bad breath gas level and the sensed temperature level may be provided, and guide information about the bad breath gas level caused by what the user has eaten (S132). Further, when the guide information about the bad breath level is provided, body temperature information about a body temperature level and a guide level may be provided.

When the fluorescence loss of the captured image is lower than 5%, guide information about the bad breath level caused by germs along with what the user has eaten, while displaying information for guiding healthy teeth (S131).

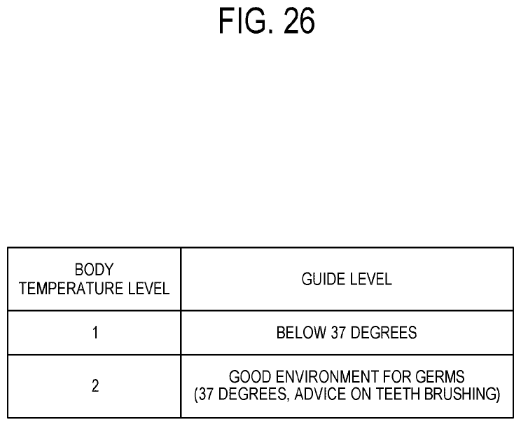

FIG. 26 is a table of showing guide information provided to a user in the user terminal 10 by measuring an oral temperature level of the user in the oral care apparatus 20 according to one embodiment of the present invention.

The table shows references of guide information about a user's body temperature level. A body temperature level of ‘1’ indicates information that a measured temperature is lower than a body temperature and has an effect on decreasing the bad breath. A body temperature level of ‘2’ indicates information that teeth-brushing is advised since the measured temperature is higher than the body temperature or the body temperature level 1 and makes a good environment for germs. When this table is provided, a part corresponding to the currently measured level may be highlighted. Thus, a user can be informed of his/her temperature level and take care of the oral cavity based on the guide information.

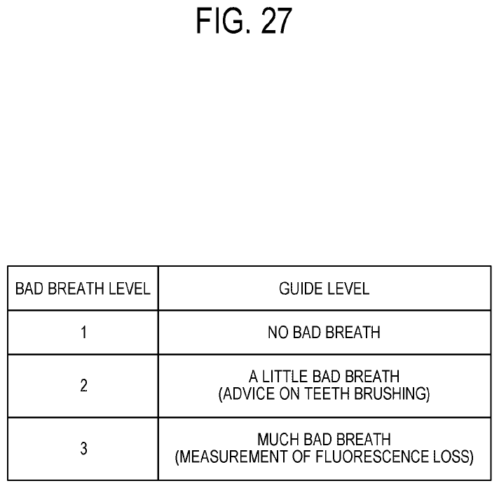

FIG. 27 is a table of showing guide information provided to a user in the user terminal 10 by measuring a bad breath level of the user in the oral care apparatus 20 according to one embodiment of the present invention.

The table shows references of guide information about a user's gas level. A bad breath level of ‘1’ indicates information that the user has little bad breath. A bad breath level of ‘2’ indicates information that the user has a little bad breath. Thus, guide information that advises brushing teeth may be displayed. A bad breath level of ‘3’ indicates information that guide information is displayed to advise measuring fluorescence loss with the first light since the user has a bad breath. When this table is provided, a part corresponding to the currently measured level may be highlighted. Thus, a user can be informed of his/her temperature level and take care of the oral cavity based on the guide information.

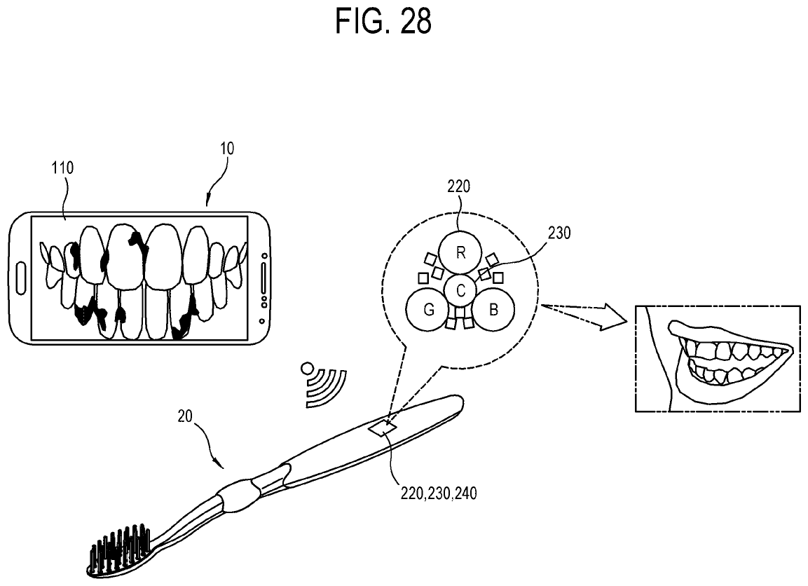

FIG. 28 illustrates an example that analysis information is provided by measuring a user's bad breath, oral temperature and light through the user terminal 10 and the oral care apparatus 20 according to one embodiment of the present invention.

The user terminal 10 of FIG. 28 may output the first light from the first region on the display 110, or the oral care apparatus 20 may output the first light from the light emitter 220. FIG. 28 shows an embodiment that the light emitter 220 of the oral care apparatus 20 outputs the first light. The first light may be output from the light emitter 220 of the oral care apparatus 20 to a user's oral cavity thoroughly, and analysis information obtained by analyzing a captured image may be displayed on the display 110 of the user terminal 10. This case may be more convenient for a user than the case of using only the user terminal 10 to analyze the oral cavity and get the analysis information.

The user terminal 10 and the oral care apparatus 20 may exchange various pieces of information through their communication modules (based on Bluetooth, Wi-Fi). The oral care apparatus 20 may be provided to have a toothbrush shape. In a grip part, the light emitter 220, the image capturer 230, and the sensor 240 are provided to emit the first light, capture an image, and sense a user's bad breath and body temperature in the user's oral cavity. Such pieces of information sensed by the oral care apparatus 20 may be transmitted to the user terminal 10. The user terminal 10 may transmit a control signal so that the first light can be output to capture an image and the bad breath or body temperature can be sensed.

Referring to FIG. 28, the oral care apparatus 20 outputs the first light to a user's oral cavity and transmits a captured image to the user terminal 10, and the user terminal 10 analyzes the captured image and displays the analysis information on the display 110.

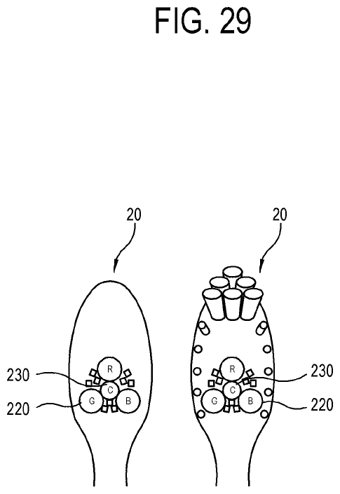

FIGS. 29 to 31 illustrate examples of showing the mounting positions and shapes of the light emitter 220, the image capturer 230 and the sensor 240 in the oral care apparatus 20 according to one embodiment of the present invention.

FIG. 29 shows that the oral care apparatus 20 is shaped like a toothbrush, in which the light emitter 220, the image capturer 230 and the sensor 240 may be provided in the front and back of a brush part.

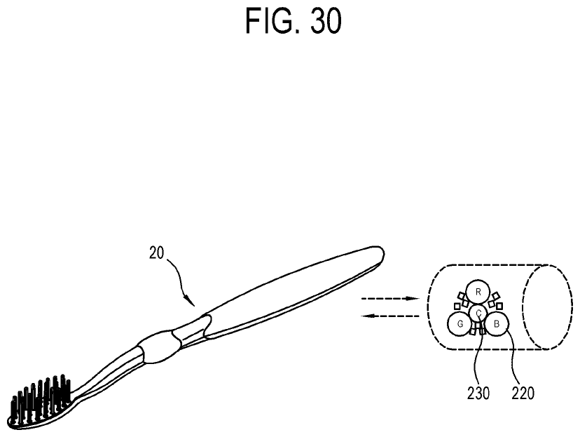

FIG. 30 shows that the light emitter 220, the image capturer 230 and the sensor 240 are detachably provided in the oral care apparatus 20. With this detachable structure, the oral care apparatus 20 may be usually used as the toothbrush without the light emitter 220, the image capturer 230 and the sensor 240, and may be used to determine the state of the oral cavity with the light emitter 220, the image capturer 230 and the sensor 240 attached thereto.

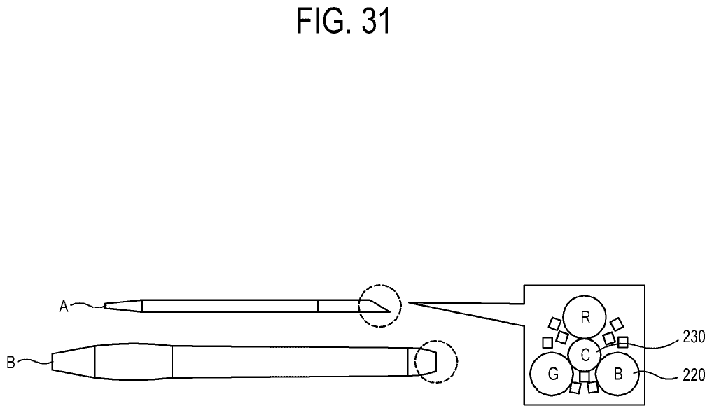

FIG. 31 shows that the oral care apparatus 20 is shaped like a pen detachably provided in the user terminal 10.

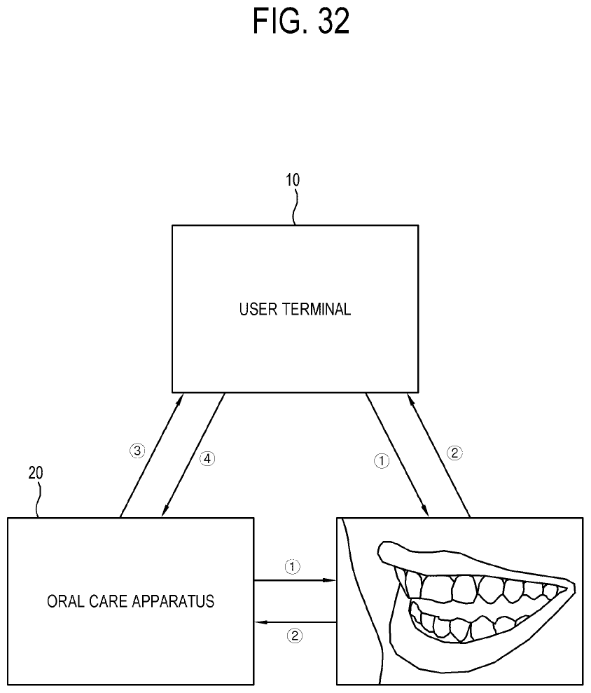

FIG. 32 illustrates an example of showing a signal flow in measuring a user's bad breath, oral temperature and light through the user terminal 10 and the oral care apparatus 20 according to one embodiment of the present invention and providing analysis information,

The user terminal 10 may independently perform the operations of FIG. 25. However, FIG. 32 shows that both the user terminal 10 and the oral care apparatus 20 are used to perform the operations of FIG. 25.

A user selects and executes an application program for determining a state of a user's oral cavity on the screen of the display 110 in the user terminal 10. As the application program is executed, the user terminal 10 transmits a control signal to the oral care apparatus 20 so as to sense a user's bad breath (\textcircled{4}). The user breathes out his/her breath toward the sensor 240 of the oral care apparatus 20 (\textcircled{1}). Thus, the gas sensor 244 of the oral care apparatus 20 senses gas of bad breath (\textcircled{2}). The controller 200 of the oral care apparatus 20 may determine whether a sensed gas level is equal to or higher than a preset level, and transmit the sensed gas level to the user terminal 10 (\textcircled{3}). When the sensed gas level is transmitted to the user terminal 10, the user terminal 10 determines whether the received gas level is equal to or higher than the preset level, and transmits a control signal to the oral care apparatus 20 in order to sense the temperature in the oral cavity when the received gas level is equal to or higher than the preset level (\textcircled{4}). Thus, a user puts the sensor of the oral care apparatus 20 into the oral cavity and senses a body temperature in the oral cavity. The controller 200 of the oral care apparatus 20 may determine whether the sensed temperature level is equal to or higher than a preset level, and transmit the sensed temperature level to the user terminal 10 (\textcircled{3}). When the sensed temperature level is transmitted to the user terminal 10, the user terminal 10 determines whether the received temperature level is equal to or higher than the preset level, and transmits a control signal to the oral care apparatus 20 so as to output the first light to the oral cavity and capture an image when the received temperature level is equal to or higher than the preset level (\textcircled{4}). Thus, the user makes the image be captured with the output first light by the image capturer 230 of the oral care apparatus 20 facing toward the oral cavity. The captured image may be transmitted to the user terminal 10 (\textcircled{3}). The user terminal 10 may analyze the received image and display analysis information on the screen of the display 110.

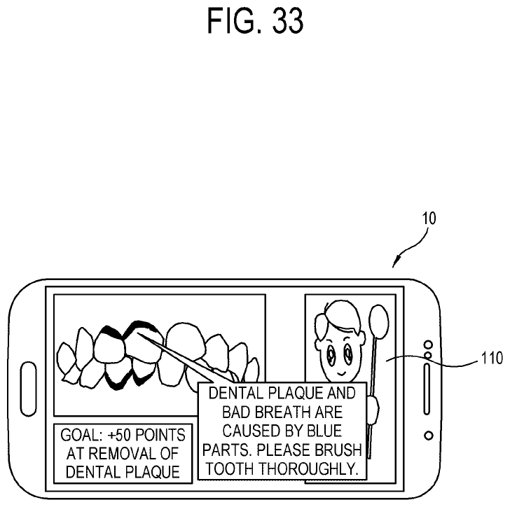

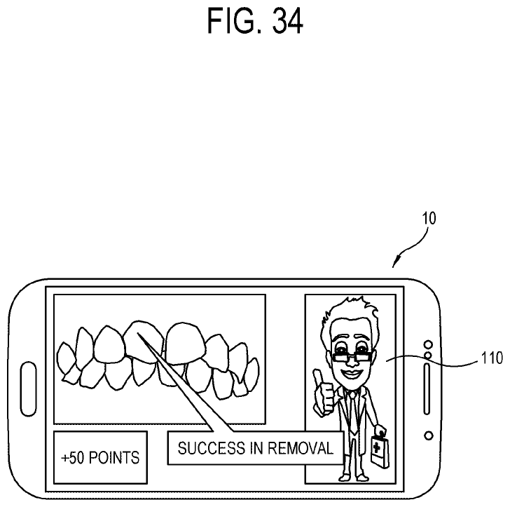

FIGS. 33 and 34 illustrate examples of conducting a dental health education and coaching through an animation or a user set character for comparison between before and after brushing teeth with respect to measured results. Further, challenge and attainment guides are provided to a user after setting a goal, thereby conducting the dental health education and coaching.

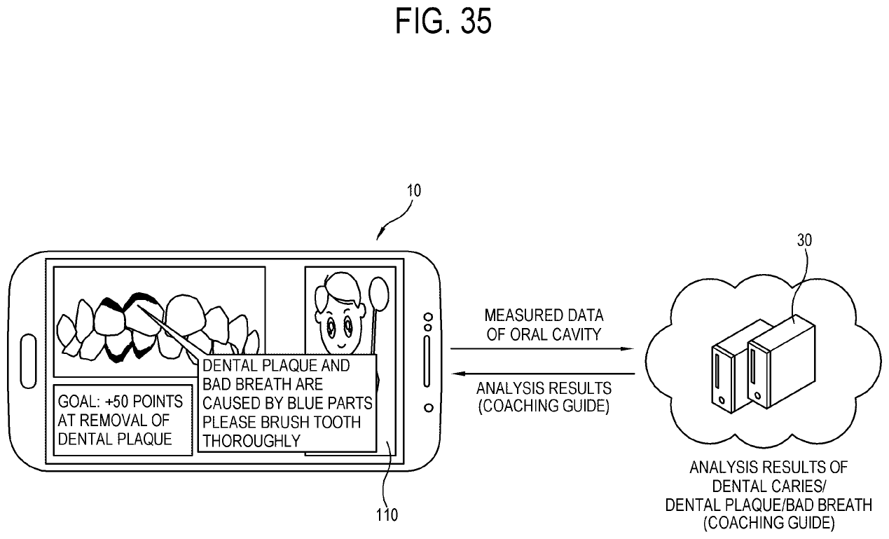

FIG. 35 illustrates an extended example that measured results for dental care are transmitted to a server capable of analyzing the measured results and services like those of FIGS. 33 and 34 are received through the server 30.

The user terminal 10 captures the first light from the first region and transmits captured data to the server 30, and the server 30 performs analysis and transmits analysis information to the user terminal 10 so that the user terminal 10 can display the received analysis information on the display 110. With regard to the bad breath and the oral temperature, the user terminal 10 may perform measurement, transmit the measured data to the server 30, receive information analyzed in the server 30, and display the analysis information on the display 110.

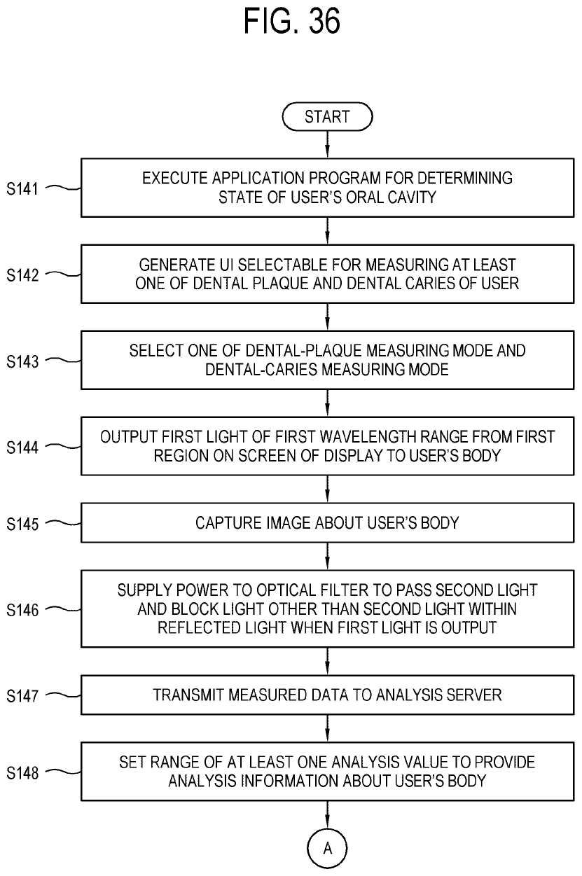

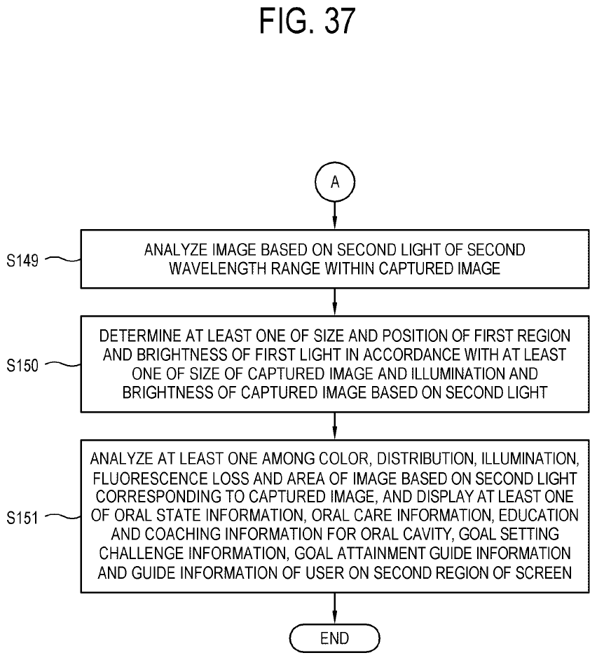

FIGS. 36 and 37 are flowcharts of showing a control method of the user terminal 10 according to one embodiment of the present invention with regard to FIGS. 33 and 34.

A user selects and executes an application program for determining a state of a user's oral cavity from the screen of the display 110 (S141).

The controller 100 control the UI generator 160 to generate a UI selectable for measuring at least one among the dental plaque, dental caries, bad breath, and oral cavity temperature of the user (S142).

The user selects one of a generated dental-plaque measuring mode UI and a generated dental-caries measuring mode UI (S143).

The controller 100 controls the display 110 so that the first region on the screen of the display 110 can output the first light having a first wavelength range to the user's body in accordance with the selected mode (S144).

The image capturer 120 captures an image with regard to the user's body (S145).

The power supply 170 supplies power to the optical filter 180 to pass the second light of the reflected light but blocks the light other than the second light when the first light is output (S146).

The data measured using the optical filter 180 is transmitted to an analysis sever (S147), thereby conducting analysis.

To provide analysis information about the user's body, a range of at least one analysis value is set (S148). The range of the analysis value maybe previously set by a user or with reference to a doctor's advice.

The image based on the second light having a second wavelength range is analyzed within the captured image (S149).

The controller 100 determines at least one of the size and position of the first region and the brightness of the first light in accordance with at least one of the size of the captured image and the illumination and brightness of the captured image based on the second light (S150).

The controller 100 controls the display 110 to have the determined size and positions of the first region and the determined brightness of the first light.

The controller 100 analyzes at least one among the color, distribution, illumination, fluorescence loss and area of the image based on the second light corresponding to the captured image, and displays at least one of the oral state information, oral care information, education and coaching information for the oral cavity, goal setting challenge information, goal attainment guide information and guide information of the user on the second region of the screen (S151). For instance, the image of the user's oral cavity may be displayed with colors to show a degree of bad breath, dental caries or dental plaque; the analysis information may be represented with digits; vibration or alarm may be given in accordance with the degrees of dental caries, dental plaque and bad breath; and the image based on the colors may be displayed with a table in which the analysis information is tabulated with digits.

The first light may be output from not the display apparatus 10 but an external device, and the control flow may be performed by receiving an image captured with regard to a region to which the first light having the first wavelength range is output. The control flow is merely one embodiment, and there are no limits to this embodiment.

In addition to the control flow, the controller 100 may measure a gas level in the user's oral cavity and measure a temperature level when the gas level is equal to or higher than a predetermined level. The controller 100 may measure the temperature level in the user's oral cavity and capture an image by outputting the first light when the temperature level is lower than or equal to a predetermined level. When the gas level in the user's oral cavity is lower than or equal to a predetermined level, the controller 100 may control the guide information to be displayed for guiding the measured gas level, i.e. a degree of bad breath without outputting the first light and capturing the image. When the temperature level in the user's oral cavity is equal to or higher than a predetermined level, the controller 100 may control the guide information to be displayed for guiding the measured temperature level and a bad breath level without outputting the first light and capturing the image.

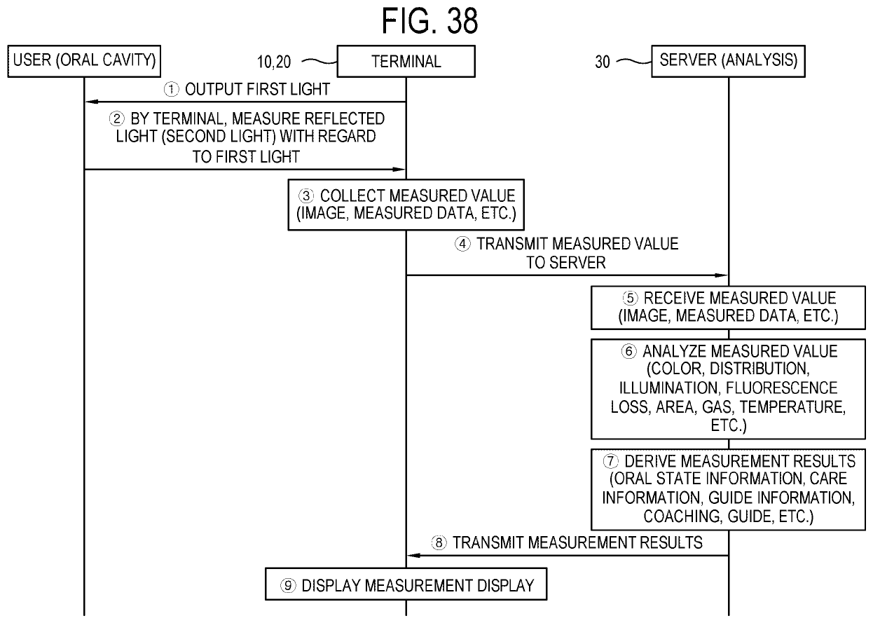

FIG. 38 illustrates an example of showing operations for providing analysis information about teeth through the user terminal 10 and the server 30 according to another embodiment of the present invention.

\textcircled{1} the user terminal 10 or the oral care apparatus 20 outputs the first light to a user's teeth.

\textcircled{2} the reflected light (i.e. second light) based on the first light is measured in the terminal.

\textcircled{3} the user terminal 10 collects images, measured data, etc. When the oral care apparatus 20 outputs the first light, the image, the measured data, etc. may be transmitted from the oral care apparatus 20 to the user terminal 10 or the server 30.

\textcircled{4} the user terminal 10 transmits the collected image, measured data, etc. to the server 30.

\textcircled{5} the server 30 receives the image, measured data, etc. from the oral care apparatus 20 or the user terminal 10.

\textcircled{6} the server 30 together with the user terminal 10 analyses the image, measured data, etc. with respect to color, distribution, illumination, fluorescence loss, area, gas, temperature, etc.

\textcircled{7} the server 30 obtains measured results such as oral state information, care information, education and coaching information for the oral cavity, goal setting challenge information, goal attainment guide information, guide information, etc.

\textcircled{8} the server 30 transmits the obtained measured results to the user terminal 10.

\textcircled{9} the user terminal 10 displays the received measured results to the display 110.

The user terminal 10 outputs light of a specific range from its screen to a user's body, captures an image with reflected light, and provides analysis information about the user's body on the screen, thereby allowing a user to easily obtain information about the state and diagnosis of his/her own teeth through the user terminal 10.

Although the present invention has been described with the limited and exemplary embodiments and drawings, the present invention is not limited to the foregoing exemplary embodiments, and various changes and modifications can be made from these descriptions by a person having an ordinary skilled in the art to which the present invention pertains. Therefore, the scope of the invention has to be defined in the appended claims and their equivalents without limitations to the described exemplary embodiments.

1. A user terminal comprising:
a display comprising a screen on which an image is displayable, and configured to output light having a specific wavelength from at least a partial region of the screen;
an image capturer configured to capture an image of a user's body; and
a controller configured to control the display to output first light of a first wavelength range from a first region on the screen, control the image capturer to capture an image, analyze the captured image, and control the display to display analysis information on a second region of the screen.

2. The user terminal according to claim 1, further comprising a sensor configured to sense at least one of gas and temperature in the user's oral cavity,
wherein the controller controls the display to output the first light based on a level of at least one of the gas and temperature sensed by the sensor.

3. The user terminal according to claim 1, wherein the controller analyzes an image based on second light of a second wavelength range within the captured image.

4. The user terminal according to claim 3, wherein the controller analyzes at least one of color, distribution, illumination, fluorescence loss and area of the image based on the second light in accordance with the captured image.

5. The user terminal according to claim 3, further comprising:
a power supply configured to supply power; and
an optical filter configured to receive power from the power supply and operate to pass the second light but block light other than the second light of the reflected light caused by the first light.

6. The user terminal according to claim 5, wherein the controller controls the power supply so that the optical filter can start the operations when the first light is output.

7. The user terminal according to claim 3, wherein the controller controls at least one among a size and a position of the first region and brightness of the first light to be varied depending on at least one among a size, illumination and brightness of the image captured with the second light.

8. The user terminal according to claim 1, further comprising:
a storage configured to store an application program for determining a state of a user's oral cavity; and
a user interface (UI) generator configured to generate a UI for the application program,
wherein the controller executes the application program, and controls the UI generator to generate a UI selectable for measuring at least one among dental plaque, dental caries, bad breath and oral temperature of the user.

9. A method of controlling a user terminal, the method comprising:
controlling a first region on a screen of a display to output first light of a first wavelength range;
capturing an image;
analyzing the captured image; and
displaying analysis information in a second region on the screen.

10. The method according to claim 9, wherein the controlling of the output of the first light comprises controlling the first light to be output based on a level of at least one of gas and temperature sensed in a user's oral cavity.

11. The method according to claim 9, wherein the analyzing of the captured image comprises analyzing an image based on second light of a second wavelength range within the captured image.

12. The method according to claim 11, wherein the displaying of the analysis information comprises analyzing at least one of color, distribution, illumination, fluorescence loss and area of the image based on the second light in accordance with the captured image.

13. An oral care apparatus comprising:
a communicator configured to communicate with a user terminal;
a light emitter configured to output light having a specific wavelength to a user's oral cavity;
an image capturer configured to capture an image of the user's oral cavity; and
a controller configured to control the light emitter to output first light of a first wavelength range, control the image capturer to capture an image, and control the communicator to transmit the captured image to the user terminal.

14. A method of controlling an oral care apparatus, the method comprising:
outputting first light of a first wavelength range;
capturing an image; and
transmitting the captured image to a user terminal.
